(19) United States
(12) Patent Application Publication
(10) Pub. No.: US 2022/0152571 A1
(43) Pub. Date: May 19, 2022

(54) POLY(AMIDE-ESTER) MICROCAPSULES

(71) Applicant: FIRMENICH SA, Satigny (CH)

(72) Inventors: Lahoussine Ouali, Satigny (CH); Damien Berthier, Satigny (CH); Marlène Jacquemond, Satigny (CH); Anaick NIcolae, Satigny (CH); Amal Elabbadi, Satigny (CH)

(21) Appl. No.: 17/439,346

(22) PCT Filed: Jul. 31, 2020
(86) PCT No.: PCT/EP2020/071630

§ 371 Date: Sep. 14, 2021

(30) Foreign Application Priority Data
Aug. 5, 2019 (EP) 19190062.0

(51) Int. Cl. B01J 13/16; C08G 69/44; C08K 9/10; C08K 5/1545; A61Q 13/00; A61K 8/11; C11D 3/37; C11D 3/50; C11D 17/00; A61K 8/88; A61Q 19/10; A61Q 15/00; A61Q 5/12; A61Q 5/02

(52) U.S. Cl. CPC B01J 13/16; A61K 2800/56; C08K 9/10; C08K 5/1545; A61Q 13/00; A61K 8/11; C11D 3/3719; C11D 3/505; C11D 17/0039; A61K 8/88; A61Q 19/10; A61Q 15/00; A61Q 5/12; A61Q 5/02; C08K 2201/007; A61K 2800/412; C08G 69/44

(57) ABSTRACT

Described herein are a process for the preparation of poly(amide-ester) microcapsules, and such poly(amide-ester) microcapsules. Perfuming compositions and consumer products comprising such capsules, in particular perfumed consumer products in the form of home care or personal care products, are also described.

### TECHNICAL FIELD

The present invention relates to a new process for the preparation of poly(amide-ester) microcapsules. Poly(amide-ester) microcapsules are also an object of the invention. Perfuming compositions and consumer products comprising said capsules, in particular perfumed consumer products in the form of home care or personal care products, are also part of the invention.

### BACKGROUND OF THE INVENTION

One of the problems faced by the perfumery industry lies in the relatively rapid loss of olfactive benefit provided by odoriferous compounds due to their volatility, particularly that of “top-notes”. In order to tailor the release rates of volatiles, delivery systems such as microcapsules containing a perfume are needed to protect and later release the core payload when triggered. A key requirement from the industry regarding these systems is to survive suspension in challenging bases without physically dissociating or degrading. This is referred to as stability for the delivery system. For instance, fragranced personal and household cleansers containing high levels of aggressive surfactant detergents are very challenging for the stability of microcapsules.

Polyurea and polyurethane-based microcapsule slurry are widely used for example in perfumery industry as they provide a long lasting pleasant olfactory effect after their applications on different substrates. Those microcapsules have been widely disclosed in the prior art (see for example WO2007/004166 or EP 2300146 from the Applicant).

Therefore, there is still a need to use alternative microcapsules, while not compromising on the performance of the microcapsules, in particular in terms of stability in a challenging medium such as a consumer product base, as well as in delivering a good performance in terms of active ingredient delivery, e.g. olfactive performance in the case of perfuming ingredients.

The present invention is proposing a solution to the above-mentioned problem, by providing a new poly(amide-ester) microcapsules.

### SUMMARY OF THE INVENTION

It has now been surprisingly found, that performing core-shell microcapsules encapsulating hydrophobic material(s) could be obtained by reacting an acyl chloride and a polyol with at least one amino compound during the interfacial polymerization. The process of the invention therefore provides a solution to the above-mentioned problems as it allows preparing microcapsules with the desired stability in challenging bases.

In a first aspect, the present invention relates to a process for preparing a core-shell poly(amide-ester) microcapsule slurry comprising the following steps:

a) Dissolving at least one acyl chloride in a hydrophobic material, preferably a perfume to form an oil phase;
b) Dispersing the oil phase obtained in step a) into a water phase comprising optionally an amino compound A or a base to form an oil-in-water emulsion;
c) Adding to the oil-in-water emulsion obtained in step b) an amino compound B
d) Performing a curing step to form poly(amide-ester) microcapsule in the form of a slurry, wherein a stabilizer is added in step a) and/or in step b), and wherein a polyol is added in step a) and/or in step b) and/or in step c).

A second object of the invention is poly(amide-ester) core-shell microcapsule comprising:
an oil based core comprising a hydrophobic material, preferably a perfume, and
a poly(amide-ester) shell.

A third object of the invention is poly(amide-ester) core-shell microcapsule slurry having at least one poly(amide-ester) core-shell microcapsule, wherein said poly(amide-ester) core-shell microcapsule comprises:
an oil-based core comprising a hydrophobic material, preferably a perfume, and
a poly(amide-ester) shell.

A fourth object of the invention is a poly(amide-ester) core-shell microcapsule slurry obtainable by the process as defined above.

A perfuming composition comprising:
(i) microcapsule slurry or microcapsules as defined above, wherein the hydrophobic material comprises a perfume,
(ii) at least one ingredient selected from the group consisting of a perfumery carrier and a perfumery base
(iii) optionally at least one perfumery adjuvant, is another object of the invention.

Another object of the invention is a consumer product comprising:
a personal care active base, and
microcapsules or microcapsule slurry as defined above or the perfuming composition as defined above,

wherein the consumer product is in the form of a personal care composition.

Another object of the invention is a consumer product comprising:
a home care or a fabric care active base, and
microcapsules or microcapsule slurry as defined above or the perfuming composition as defined above,

wherein the consumer product is in the form of a home care or a fabric care composition.

### DETAILED DESCRIPTION OF THE INVENTION

Unless stated otherwise, percentages (%) are meant to designate a percentage by weight of a composition.

By “hydrophobic material”, it is meant any hydrophobic material—single material or a mixture of material—which forms a two-phase dispersion when mixed with water.

By “ingredient”, it is meant a single compound or a combination of ingredients.

By “perfume or flavor oil”, it is meant a single perfuming or flavoring compound or a mixture of several perfuming or flavoring compounds.

By “consumer product” or “end-product” it is meant a manufactured product ready to be distributed, sold and used by a consumer.

By “polyamide(ester)-based microcapsule” it is meant that the polymer comprises both amide linkages and ester linkages, amide linkages and ester linkage being respectively produced by the amino groups of the amino compound and by the polyol capable of further reacting with the acyl chloride.

By “polyol”, it is meant a compound having at least two hydroxyl functions.

For the sake of clarity, by the expression “dispersion” in the present invention it is meant a system in which particles are dispersed in a continuous phase of a different composition and it specifically includes a suspension or an emulsion.

A “microcapsule”, or the similar, in the present invention it is meant that core-shell microcapsules have a particle size distribution in the micron range (e.g. a mean diameter (d(v, 0.5)) comprised between about 1 and 3000 microns, preferably comprised between 1 and 1000 microns, more preferably between 1 and 500 microns, and even more preferably between 5 and 50 microns) and comprise an external solid polymer-based shell and an internal continuous oil phase enclosed by the external shell.

By “microcapsule slurry”, it is meant microcapsule(s) that is (are) dispersed in a liquid.

According to an embodiment, the slurry is an aqueous slurry, i.e the microcapsule(s) is (are) dispersed in an aqueous phase.

By “amino-compound” it should be understood a compound having at least two reactive amine groups.

In the present invention, the terms “acyl chloride” and “acid chloride” are used indifferently.

It has been found that core-shell poly(amide-ester) microcapsules with overall good performance in challenging bases could be obtained when an acyl chloride and a polyol react with at least one amino-compound during the interfacial polymerization.

### DESCRIPTION OF DRAWINGS

FIG. 1 represents a SEM picture of microcapsules according to the invention.

FIGS. 2 and 3 represent a TGA measurement of microcapsules according to the invention.

### PROCESS FOR PREPARING A POLY(AMIDE-ESTER) MICROCAPSULE SLURRY

A first object of the invention is therefore a process for preparing a core-shell poly(amide-ester) microcapsule slurry comprising the following steps:

a) Dissolving at least one acyl chloride in a hydrophobic material, preferably a perfume to form an oil phase;
b) Dispersing the oil phase obtained in step a) into a water phase comprising optionally an amino compound A or a base to form an oil-in-water emulsion;
c) Adding to the oil-in-water emulsion obtained in step b) an amino compound B
d) Performing a curing step to form poly(amide-ester) microcapsule in the form of a slurry,
wherein a stabilizer is added in step a) and/or in step b), and
wherein a polyol is added in step a) and/or in step b) and/or in step c).

According to an embodiment, the polyol is added in the oil phase.

In one step of the process, an oil phase is formed by admixing at least one hydrophobic material with at least one acyl chloride.

According to an embodiment, the acyl chloride is chosen in the group consisting of benzene-1,2,4-tricarbonyl trichloride, benzene-1,2,4,5-tetracarbonyl tetrachloride, cyclohexane-1,3,5-tricarbonyl trichloride, isophthalyol dichloride, diglycolyl dichloride, terephthaloyl chloride, succinic dichloride, and mixtures thereof

According to a particular embodiment, the acyl chloride is 1,3,5-benzene tricarbonyl chloride.

According to a particular embodiment, the acyl chloride has the following formula (I)

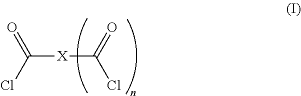

wherein n is an integer varying between 1 and 8, preferably between 1 and 6, more preferably between 1 and 4, and
wherein X is either an (n+1)-valent C₃ to C₆ alkyl group, or an (n+1)-valent C₂ to C₄₅ hydrocarbon group comprising at least one group selected from (i) to (vi),

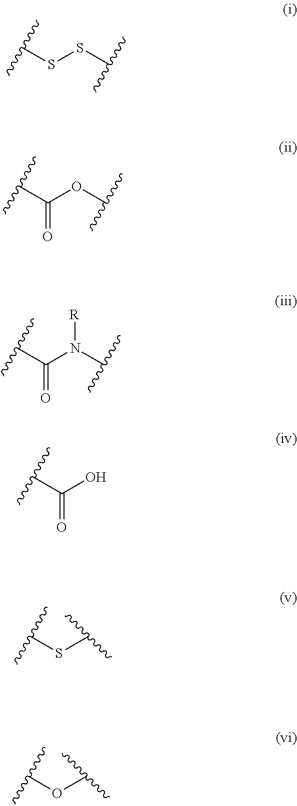

wherein R is a hydrogen atom or a methyl or ethyl group, preferably a hydrogen atom.

According to an embodiment, if the hydrocarbon group X comprises several groups selected from (i) to (vi), they are each separated by at least one carbon atom of X.

It is understood that by “ . . . hydrocarbon group . . . ” it is meant that said group consists of hydrogen and carbon atoms and can be in the form of an aliphatic hydrocarbon, i.e. linear or branched saturated hydrocarbon (e.g. alkyl group), a linear or branched unsaturated hydrocarbon (e.g. alkenyl or alkynil group), a saturated cyclic hydrocarbon (e.g. cycloalkyl) or an unsaturated cyclic hydrocarbon (e.g. cycloalkenyl or cycloalkynyl), or can be in the form of an aromatic hydrocarbon, i.e. aryl group, or can also be in the form of a mixture of said type of groups, e.g. a specific group may comprise a linear alkyl, a branched alkenyl (e.g. having one or more carbon-carbon double bonds), a (poly)cycloalkyl and an aryl moiety, unless a specific limitation to only one type is mentioned. Similarly, in all the embodiments of the invention, when a group is mentioned as being in the form of more than one type of topology (e.g. linear, cyclic or branched) and/or being saturated or unsaturated (e.g. alkyl, aromatic or alkenyl), it is also meant a group which may comprise moieties having any one of said topologies or being saturated or unsaturated, as explained above. Similarly, in all the embodiments of the invention, when a group is mentioned as being in the form of one type of saturation or unsaturation, (e.g. alkyl), it is meant that said group can be in any type of topology (e.g. linear, cyclic or branched) or having several moieties with various topologies.

It is understood that with the term “ . . . a hydrocarbon group, possibly comprising . . . ” it is meant that said hydrocarbon group optionally comprises heteroatoms to form ether, thioether, amine, nitrile or carboxylic acid groups. These groups can either substitute a hydrogen atom of the hydrocarbon group and thus be laterally attached to said hydrocarbon, or substitute a carbon atom (if chemically possible) of the hydrocarbon group and thus be inserted into the hydrocarbon chain or ring.

According to an embodiment, when group (vi) is present, it is only present in combination with either one of groups (i) to (v).

According to a particular embodiment, the acyl chloride is chosen from the group consisting of propane-1,2,3-tricarbonyl trichloride, cyclohexane-1,2,4,5-tetracarbonyl tetrachloride, 2,2′-disulfanediyldisuccinyl dichloride, 2-(2-chloro-2-oxo-ethyl)sulfanylbutanedioyl dichloride, (4-chloro-4-oxobutanoyl)-L-glutamoyl dichloride, (S)-4-((1,5-dichloro-1,5-dioxopentan-2-yl)amino)-4-oxobutanoic acid, 2,2-bis[(4-chloro-4-oxo-butanoyl)oxymethyl]butyl 4-chloro-4-oxo-butanoate, [2-[2,2-bis[(4-chloro-4-oxo-butanoyl)oxymethyl]butoxymethyl]-2-[(4-chloro-4-oxo-butanoyl)oxymethyl]butyl] 4-chloro-4-oxo-butanoate, 2,2-bis[(2-chlorocarbonylbenzoyl)oxymethyl]butyl 2-chlorocarbonyl-benzoate, [2-[2,2-bis[(2-chlorocarbonylbenzoyl)oxymethyl]butoxymethyl]-2-[(2-chlorocarbonylbenzoyl)oxymethyl]butyl] 2-chlorocarbonylbenzoate, 4-(2,4,5-trichlorocarbonylbenzoyl)oxybutyl 2,4,5-trichlorocarbonyl-benzoate, and mixtures thereof.

The weight ratio between the acyl chloride and the hydrophobic material is preferably comprised between 0.01 and 0.09, more preferably between 0.03 and 0.07.

The acyl chloride can be dissolved directly in the hydrophobic material or can be pre-dispersed in an inert solvent such as benzyl benzoate before mixing with the hydrophobic material, preferably a perfume oil.

According to a particular embodiment, a polyisocyanate having at least two isocyanate functional groups is added in the oil phase.

Suitable polyisocyanates used according to the invention include aromatic polyisocyanate, aliphatic polyisocyanate and mixtures thereof. Said polyisocyanate comprises at least 2, preferably at least 3 but may comprise up to 6, or even only 4, isocyanate functional groups. According to a particular embodiment, a triisocyanate (3 isocyanate functional group) is used.

According to one embodiment, said polyisocyanate is an aromatic polyisocyanate.

The term “aromatic polyisocyanate” is meant here as encompassing any polyisocyanate comprising an aromatic moiety. Preferably, it comprises a phenyl, a toluyl, a xylyl, a naphthyl or a diphenyl moiety, more preferably a toluyl or a xylyl moiety. Preferred aromatic polyisocyanates are biurets, polyisocyanurates and trimethylol propane adducts of diisocyanates, more preferably comprising one of the above-cited specific aromatic moieties. More preferably, the aromatic polyisocyanate is a polyisocyanurate of toluene diisocyanate (commercially available from Bayer under the tradename Desmodur® RC), a trimethylol propane-adduct of toluene diisocyanate (commercially available from Bayer under the tradename Desmodur® L75), a trimethylol propane-adduct of xylylene diisocyanate (commercially available from Mitsui Chemicals under the tradename Takenate® D-110N). In a most preferred embodiment, the aromatic polyisocyanate is a trimethylol propane-adduct of xylylene diisocyanate.

According to another embodiment, said polyisocyanate is an aliphatic polyisocyanate. The term “aliphatic polyisocyanate” is defined as a polyisocyanate which does not comprise any aromatic moiety. Preferred aliphatic polyisocyanates are a trimer of hexamethylene diisocyanate, a trimer of isophorone diisocyanate, a trimethylol propane-adduct of hexamethylene diisocyanate (available from Mitsui Chemicals) or a biuret of hexamethylene diisocyanate (commercially available from Bayer under the tradename Desmodur® N 100), among which a biuret of hexamethylene diisocyanate is even more preferred.

According to another embodiment, the at least one polyisocyanate is in the form of a mixture of at least one aliphatic polyisocyanate and of at least one aromatic polyisocyanate, both comprising at least two or three isocyanate functional groups, such as a mixture of a biuret of hexamethylene diisocyanate with a trimethylol propane-adduct of xylylene diisocyanate, a mixture of a biuret of hexamethylene diisocyanate with a polyisocyanurate of toluene diisocyanate and a mixture of a biuret of hexamethylene diisocyanate with a trimethylol propane-adduct of toluene diisocyanate. Most preferably, it is a mixture of a biuret of hexamethylene diisocyanate with a trimethylol propane-adduct of xylylene diisocyanate. Preferably, when used as a mixture the molar ratio between the aliphatic polyisocyanate and the aromatic polyisocyanate is ranging from 80:20 to 10:90.

According to an embodiment, the at least one polyisocyanate used in the process of the invention is present in amounts representing from 0.1 to 15%, preferably from 0.5 to 10% and more preferably from 0.8 to 6%, and even more preferably between 1 and 3% by weight based on the total amount of the oil phase.

### Polyol

Examples of polyols that can be used in the present invention are triethanolamine, di(trimethylolpropane), ethylene glycol, glycerol, 1,4-butanediol, 1,2-hexanediol, 1,6-hexanediol, 2-ethyl-2-(hydroxymethyl)propane-1,3-diol (trimethylolpropane, TMP), 2,2-bis(hydroxymethyl)propane-1,3-diol (pentaerythritol), 2-amino-2-ethylpropane-1,3-diol, 2-amino-2-(hydroxymethyl)propane-1,3-diol, 2,2′-azanediylbis(ethan-1-ol), 2-aminopropane-1,3-diol, 2-amino-2-methylpropane-1,3-diol, polyphenols or a mixture thereof.

According to an embodiment, the polyol is a non-cleavable polyol.

According to an embodiment, the polyol is not polyvinyl alcohol.

According to an embodiment, the polyol is not glycerol.

Polyphenols can be simple monocyclic phenols (such as phloroglucinol) or polymers thereof (such as condensed tannins, hydrolysable tannins).

Polyphenols can be monocyclic or polycyclic plant polyphenols such as flavonoids, isoflavonoids, neoflavonoids, gallotannins and ellagotannins, catechol and derivatives thereof such as DL-3,4-dihydroxyphenylalanine or DL-DOPA, catecholamines such as 3-hydroxytyramine or dopamine, phloroglucinol, phenolic acids such as caffeic acid, dihydrocaffeic acid, protocatechuic acid, chlorogenic acid, isochlorogenic acid, gentisic acid, homogentisic acid, gallic acid, hexahydroxydiphenic acid, ellagic acid, rosmarinic acid or lithospermic acid, phenolic acid derivatives, particularly their esters or their heterosides, curcumin, polyhydroxylated coumarins, polyhydroxylated lignans or neolignans, or a mixture containing one or more plant polyphenols or derivatives thereof, such as silymarin.

When a polyphenol is used, it is preferably added in step b) (in the water phase) and/or in step c) (once the oil-in-water emulsion is formed). When added in step c), it is preferably added before the addition of the amino compound B.

According to any one of the invention's embodiments, the polyol represents between about 0.1% and 5%, or even between 0.2% and 3%, by weight, relative to the total weight of the dispersion as obtained after step b).

The polyol can be pre-dissolved in an inert solvent such as ethyl acetate.

According to a particular embodiment, the molar ratio between the polyol and the acyl chloride is between 0.01 and 2, preferably between 0.05 and 1.5.

### Hydrophobic Material

Hydrophobic material according to the invention can be “inert” material like solvents or active ingredients.

When, hydrophobic materials are active ingredient, it is preferably chosen from the group consisting of flavor, flavor ingredients, perfume, perfume ingredients, nutraceuticals, cosmetics, pest control agents, biocide actives and mixtures thereof.

According to a particular embodiment, the hydrophobic material comprises a mixture of a perfume with another ingredient selected from the group consisting of nutraceuticals, cosmetics, pest control agents and biocide actives.

According to a particular embodiment, the hydrophobic material comprises a mixture of biocide actives with another ingredient selected from the group consisting of perfume, nutraceuticals, cosmetics, pest control agents.

According to a particular embodiment, the hydrophobic material comprises a mixture of pest control agents with another ingredient selected from the group consisting of perfume, nutraceuticals, cosmetics, biocide actives.

According to a particular embodiment, the hydrophobic material comprises a perfume.

According to a particular embodiment, the hydrophobic material consists of a perfume.

According to a particular embodiment, the hydrophobic material consists of biocide actives.

According to a particular embodiment, the hydrophobic material consists of pest control agents.

By “perfume” (or also “perfume oil”) what is meant here is an ingredient or composition that is a liquid at about 20° C. According to any one of the above embodiments said perfume oil can be a perfuming ingredient alone or a mixture of ingredients in the form of a perfuming composition. As a “perfuming ingredient” it is meant here a compound, which is used for the primary purpose of conferring or modulating an odour. In other words such an ingredient, to be considered as being a perfuming one, must be recognized by a person skilled in the art as being able to at least impart or modify in a positive or pleasant way the odor of a composition, and not just as having an odor. For the purpose of the present invention, perfume oil also includes combination of perfuming ingredients with substances which together improve, enhance or modify the delivery of the perfuming ingredients, such as perfume precursors, emulsions or dispersions, as well as combinations which impart an additional benefit beyond that of modifying or imparting an odor, such as long-lasting, blooming, malodour counteraction, antimicrobial effect, microbial stability, pest control.

The nature and type of the perfuming ingredients present in the oil phase do not warrant a more detailed description here, which in any case would not be exhaustive, the skilled person being able to select them on the basis of its general knowledge and according to intended use or application and the desired organoleptic effect. In general terms, these perfuming ingredients belong to chemical classes as varied as alcohols, aldehydes, ketones, esters, ethers, acetates, nitriles, terpenoids, nitrogenous or sulphurous heterocyclic compounds and essential oils, and said perfuming co-ingredients can be of natural or synthetic origin. Many of these co-ingredients are in any case listed in reference texts such as the book by S. Arctander, Perfume and Flavor Chemicals, 1969, Montclair, N.J., USA, or its more recent versions, or in other works of a similar nature, as well as in the abundant patent literature in the field of perfumery. It is also understood that said ingredients may also be compounds known to release in a controlled manner various types of perfuming compounds.

In particular one may cite perfuming ingredients which are commonly used in perfume formulations, such as:
Aldehydic ingredients: decanal, dodecanal, 2-methyl-undecanal, 10-undecenal, octanal, nonanal and/or nonenal;
Aromatic-herbal ingredients: eucalyptus oil, camphor, eucalyptol, 5-methyltricyclo[6.2.1.0˜2,7-]undecan-4-one, 1-methoxy-3-hexanethiol, 2-ethyl-4,4-dimethyl-1,3-oxathiane, 2,2,7/8,9/10-Tetramethylspiro[5.5]undec-8-en-1-one, menthol and/or alpha-pinene;
Balsamic ingredients: coumarin, ethylvanillin and/or vanillin;
Citrus ingredients: dihydromyrcenol, citral, orange oil, linalyl acetate, citronellyl nitrile, orange terpenes, limonene, 1-p-menthen-8-yl acetate and/or 1,4(8)-p-menthadiene;
Floral ingredients: methyl dihydrojasmonate, linalool, citronellol, phenylethanol, 3-(4-tert-butylphenyl)-2-methylpropanal, hexylcinnamic aldehyde, benzyl acetate, benzyl salicylate, tetrahydro-2-isobutyl-4-methyl-4(2H)-pyranol, beta ionone, methyl 2-(methylamino)benzoate, (E)-3-methyl-4-(2,6,6-trimethyl-2-cyclohexen-1-yl)-3-buten-2-one, (1E)-1-(2,6,6-trimethyl-2-cyclohexen-1-yl)-1-penten-3-one, 1-(2,6,6-trimethyl-1,3-cyclohexadien-1-yl)-2-buten-1-one, (2E)-1-(2,6,6-trimethyl-2-cyclohexen-1-yl)-2-buten-1-one, (2E)-1-[2,6,6-trimethyl-3-cyclohexen-1-yl]-2-buten-1-one, (2E)-1-(2,6,6-trimethyl-1-cyclohexen-1-yl)-2-buten-1-one, 2,5-dimethyl-2-indanmethanol, 2,6,6-trimethyl-3-cyclohexene-1-carboxylate, 3-(4,4-dimethyl-1-cyclohexen-1-yl)propanal, hexyl salicylate, 3,7-dimethyl-1,6-nonadien-3-ol, 3-(4-isopropylphenyl)-2-methylpropanal, verdyl acetate, geraniol, p-menth-1-en-8-ol, 4-(1,1-dimethylethyl)-1-cyclohexyle acetate, 1,1-dimethyl-2-phenylethyl acetate, 4-cyclohexyl-2-methyl-2-butanol, amyl salicylate, high cis methyl dihydrojasmonate, 3-methyl-5-phenyl-1-pentanol, verdyl proprionate, geranyl acetate, tetrahydro linalool, cis-7-p-menthanol, propyl (S)-2-(1,1-dimethylpropoxy)propanoate, 2-methoxynaphthalene, 2,2,2-trichloro-1-phenylethyl acetate, 4/3-(4-hydroxy-4-methylpentyl)-3-cyclohexene-1-carbaldehyde, amylcinnamic aldehyde, 8-decen-5-olide, 4-phenyl-2-butanone, isononyle acetate, 4-(1,1-dimethylethyl)-1-cyclohexyl acetate, verdyl isobutyrate and/or mixture of methylionones isomers;
Fruity ingredients: gamma-undecalactone, 2,2,5-trimethyl-5-pentylcyclopentanone, 2-methyl-4-propyl-1,3-oxathiane, 4-decanolide, ethyl 2-methyl-pentanoate, hexyl acetate, ethyl 2-methylbutanoate, gamma-nonalactone, allyl heptanoate, 2-phenoxyethyl isobutyrate, ethyl 2-methyl-1,3-dioxolane-2-acetate, 3-(3,3/1,1-dimethyl-5-indanyl)propanal, diethyl 1,4-cyclohexanedicarboxylate, 3-methyl-2-hexen-1-yl acetate, 1-[3,3-dimethylcyclohexyl]ethyl [3-ethyl-2-oxiranyl]acetate and/or diethyl 1,4-cyclohexane dicarboxylate;
Green ingredients: 2-methyl-3-hexanone (E)-oxime, 2,4-dimethyl-3-cyclohexene-1-carbaldehyde, 2-tert-butyl-1-cyclohexyl acetate, styrallyl acetate, allyl (2-methylbutoxy)acetate, 4-methyl-3-decen-5-ol, diphenyl ether, (Z)-3-hexen-1-ol and/or 1-(5,5-dimethyl-1-cyclohexen-1-yl)-4-penten-1-one;
Musk ingredients: 1,4-dioxa-5,17-cycloheptadecanedione, (Z)-4-cyclopentadecen-1-one, 3-methylcyclopentadecanone, 1-oxa-12-cyclohexadecen-2-one, 1-oxa-13-cyclohexadecen-2-one, (9Z)-9-cycloheptadecen-1-one, 2-{1S)-1-[(1R)-3,3-dimethylcyclohexyl]ethoxy}-2-oxoethyl propionate 3-methyl-5-cyclopentadecen-1-one, 1,3,4,6,7,8-hexahydro-4,6,6,7,8,8-hexamethyl-cyclopenta-g-2-benzopyrane, (1S,1′R)-2-[1-(3′,3′-dimethyl-1′-cyclohexyl)ethoxy]-2-methylpropyl propanoate, oxacyclohexadecan-2-oneand/or (1S,1′R)-[1-(3′,3′-dimethyl-1′-cyclohexyl)ethoxycarbonyl]methyl propanoate,
Woody ingredients: 1-[(1RS,6SR)-2,2,6-trimethylcyclohexyl]-3-hexanol, 3,3-dimethyl-5-[(1R)-2,2,3-trimethyl-3-cyclopenten-1-yl]-4-penten-2-ol, 3,4′-dimethylspiro[oxirane-2,9′-tricyclo[6.2.1.0^{2,7}]undec[4]ene, (1-ethoxyethoxy)cyclododecane, 2,2,9,11-tetramethylspiro[5.5]undec-8-en-1-yl acetate, 1-(octahydro-2,3,8,8-tetramethyl-2-naphtalenyl)-1-ethanone, patchouli oil, terpenes fractions of patchouli oil, Clearwood®, (1′R,E)-2-ethyl-4-(2′,2′,3′-trimethyl-3′-cyclopenten-1′-yl)-2-buten-1-ol, 2-ethyl-4-(2,2,3-trimethyl-3-cyclopenten-1-yl)-2-buten-1-ol, methyl cedryl ketone, 5-(2,2,3-trimethyl-3-cyclopentenyl)-3-methylpentan-2-ol, 1-(2,3,8,8-tetramethyl-1,2,3,4,6,7,8,8a-octahydronaphthalen-2-yl)ethan-1-one and/or isobornyl acetate;
Other ingredients (e.g. amber, powdery spicy or watery): dodecahydro-3a,6,6,9a-tetramethyl-naphtho[2,1-b]furan and any of its stereoisomers, heliotropin, anisic aldehyde, eugenol, cinnamic aldehyde, clove oil, 3-(1,3-benzodioxol-5-yl)-2-methylpropanal, 7-methyl-2H-1,5-benzodioxepin-3(4H)-one, 2,5,5-trimethyl-1,2,3,4,4a,5,6,7-octahydro-2-naphthalenol, 1-phenylvinyl acetate, 6-methyl-7-oxa-1-thia-4-azaspiro[4.4]nonan and/or 3-(3-isopropyl-1-phenyl)butanal.

It is also understood that said ingredients may also be compounds known to release in a controlled manner various types of perfuming compounds also known as properfume or profragrance. Non-limiting examples of suitable properfumes may include 4-(dodecylthio)-4-(2,6,6-trimethyl-2-cyclohexen-1-yl)-2-butanone, 4-(dodecylthio)-4-(2,6,6-trimethyl-1-cyclohexen-1-yl)-2-butanone, trans-3-(dodecylthio)-1-(2,6,6-trimethyl-3-cyclohexen-1-yl)-1-butanone, 2-phenylethyl oxo(phenyl)acetate, 3,7-dimethylocta-2,6-dien-1-yl oxo(phenyl)acetate, (Z)-hex-3-en-1-yl oxo(phenyl)acetate, 3,7-dimethyl-2,6-octadien-1-yl hexadecanoate, bis(3,7-dimethylocta-2,6-dien-1-yl) succinate, (2-((2-methylundec-1-en-1-yl)oxy)ethyl)benzene, 1-methoxy-4-(3-methyl-4-phenethoxybut-3-en-1-yl)benzene, (3-methyl-4-phenethoxybut-3-en-1-yl)benzene, 1-(((Z)-hex-3-en-1-yl)oxy)-2-methylundec-1-ene, (2-((2-methylundec-1-en-1-yl)oxy)ethoxy)benzene, 2-methyl-1-(octan-3-yloxy)undec-1-ene, 1-methoxy-4-(1-phenethoxyprop-1-en-2-yl)benzene, 1-methyl-4-(1-phenethoxyprop-1-en-2-yl)benzene, 2-(1-phenethoxyprop-1-en-2-yl)naphthalene, (2-phenethoxyvinyl)benzene, 2-(1-((3,7-dimethyloct-6-en-1-yl)oxy)prop-1-en-2-yl)naphthalene or a mixture thereof.

The perfuming ingredients may be dissolved in a solvent of current use in the perfume industry. The solvent is preferably not an alcohol. Examples of such solvents are diethyl phthalate, isopropyl myristate, Abalyn® (rosin resins, available from Eastman), benzyl benzoate, ethyl citrate, limonene or other terpenes, or isoparaffins. Preferably, the solvent is very hydrophobic and highly sterically hindered, like for example Abalyn® or benzyl benzoate. Preferably the perfume comprises less than 30% of solvent. More preferably the perfume comprises less than 20% and even more preferably less than 10% of solvent, all these percentages being defined by weight relative to the total weight of the perfume. Most preferably, the perfume is essentially free of solvent.

Preferred perfuming ingredients are those having a high steric hindrance and in particular those from one of the following groups:
Group 1: perfuming ingredients comprising a cyclohexane, cyclohexene, cyclohexanone or cyclohexenone ring substituted with at least one linear or branched C₁ to C₄ alkyl or alkenyl substituent;
Group 2: perfuming ingredients comprising a cyclopentane, cyclopentene, cyclopentanone or cyclopentenone ring substituted with at least one linear or branched C₄ to C₈ alkyl or alkenyl substituent;
Group 3: perfuming ingredients comprising a phenyl ring or perfuming ingredients comprising a cyclohexane, cyclohexene, cyclohexanone or cyclohexenone ring substituted with at least one linear or branched C₅ to C₈ alkyl or alkenyl substituent or with at least one phenyl substituent and optionally one or more linear or branched C₁ to C₃ alkyl or alkenyl substituents;
Group 4: perfuming ingredients comprising at least two fused or linked C₅ and/or C₆ rings;
Group 5: perfuming ingredients comprising a camphor-like ring structure;
Group 6: perfuming ingredients comprising at least one C7 to C20 ring structure;
Group 7: perfuming ingredients having a log P value above 3.5 and comprising at least one tert-butyl or at least one trichloromethyl substitutent;

Examples of ingredients from each of these groups are:
Group 1: 2,4-dimethyl-3-cyclohexene-1-carbaldehyde (origin: Firmenich SA, Geneva, Switzerland), isocyclocitral, menthone, isomenthone, methyl 2,2-dimethyl-6-methylene-1-cyclohexanecarboxylate (origin: Firmenich SA, Geneva, Switzerland), nerone, terpineol, dihydroterpineol, terpenyl acetate, dihydroterpenyl acetate, dipentene, eucalyptol, hexylate, rose oxide, (S)-1,8-p-menthadiene-7-ol (origin: Firmenich SA, Geneva, Switzerland), 1-p-menthene-4-ol, (1RS,3RS,4SR)-3-p-mentanyl acetate, (1R,2S,4R)-4,6,6-trimethyl-bicyclo[3,1,1]heptan-2-ol, tetrahydro-4-methyl-2-phenyl-2H-pyran (origin: Firmenich SA, Geneva, Switzerland), cyclohexyl acetate, cyclanol acetate, 1,4-cyclohexane diethyldicarboxylate (origin: Firmenich SA, Geneva, Switzerland), (3ARS,6SR,7ASR)-perhydro-3,6-dimethyl-benzo[B]furan-2-one (origin: Firmenich SA, Geneva, Switzerland), ((6R)-perhydro-3,6-dimethyl-benzo[B]furan-2-one (origin: Firmenich SA, Geneva, Switzerland), 2,4,6-trimethyl-4-phenyl-1,3-dioxane, 2,4,6-trimethyl-3-cyclohexene-1-carbaldehyde;
Group 2: (E)-3-methyl-5-(2,2,3-trimethyl-3-cyclopenten-1-yl)-4-penten-2-ol (origin: Givaudan SA, Vernier, Switzerland), (1′R,E)-2-ethyl-4-(2′,2′,3′-trimethyl-3′-cyclopenten-1′-yl)-2-buten-1-ol (origin: Firmenich SA, Geneva, Switzerland), (1′R,E)-3,3-dimethyl-5-(2′,2′,3′-trimethyl-3′-cyclopenten-1′-yl)-4-penten-2-ol (origin: Firmenich SA, Geneva, Switzerland), 2-heptylcyclopentanone, methyl-cis-3-oxo-2-pentyl-1-cyclopentane acetate (origin: Firmenich SA, Geneva, Switzerland), 2,2,5-Trimethyl-5-pentyl-1-cyclopentanone (origin: Firmenich SA, Geneva, Switzerland), 3,3-dimethyl-5-(2,2,3-trimethyl-3-cyclopenten-1-yl)-4-penten-2-ol (origin: Firmenich SA, Geneva, Switzerland), 3-methyl-5-(2,2,3-trimethyl-3-cyclopenten-1-yl)-2-pentanol (origin, Givaudan SA, Vernier, Switzerland);
Group 3: damascones, 1-(5,5-dimethyl-1-cyclohexen-1-yl)-4-penten-1-one (origin: Firmenich SA, Geneva, Switzerland), nectalactone ((1′R)-2-[2-(4′-methyl-3′-cyclohexen-1′-yl)propyl]cyclopentanone), alpha-ionone, beta-ionone, damascenone, mixture of 1-(5,5-dimethyl-1-cyclohexen-1-yl)-4-penten-1-one and 1-(3,3-dimethyl-1-cyclohexen-1-yl)-4-penten-1-one (origin: Firmenich SA, Geneva, Switzerland), 1-(2,6,6-trimethyl-1-cyclohexen-1-yl)-2-buten-1-one (origin: Firmenich SA, Geneva, Switzerland), (1S,1′R)-[1-(3′,3′-Dimethyl-1′-cyclohexyl)ethoxycarbonyl]methyl propanoate (origin: Firmenich SA, Geneva, Switzerland), 2-tert-butyl-1-cyclohexyl acetate (origin: International Flavors and Fragrances, USA), 1-(2,2,3,6-tetramethyl-cyclohexyl)-3-hexanol (origin: Firmenich SA, Geneva, Switzerland), trans-1-(2,2,6-trimethyl-1-cyclohexyl)-3-hexanol (origin: Firmenich SA, Geneva, Switzerland), (E)-3-methyl-4-(2,6,6-trimethyl-2-cyclohexen-1-yl)-3-buten-2-one, terpenyl isobutyrate, 4-(1,1-dimethylethyl)-1-cyclohexyl acetate (origin: Firmenich SA, Geneva, Switzerland), 8-methoxy-1-p-menthene, (1S,1′R)-2-[1-(3′,3′-dimethyl-1′-cyclohexyl) ethoxy]-2-methylpropyl propanoate (origin: Firmenich SA, Geneva, Switzerland), para tert-butylcyclohexanone, menthenethiol, 1-methyl-4-(4-methyl-3-pentenyl)-3-cyclohexene-1-carbaldehyde, allyl cyclohexylpropionate, cyclohexyl salicylate, 2-methoxy-4-methylphenyl methyl carbonate, ethyl 2-methoxy-4-methylphenyl carbonate, 4-ethyl-2-methoxyphenyl methyl carbonate;
Group 4: Methyl cedryl ketone (origin: International Flavors and Fragrances, USA), a mixture of (1RS,2SR,6RS,7RS,8SR)-tricyclo[5.2.1.0˜2,6˜]dec-3-en-8-yl 2-methylpropanoate and (1RS,2SR,6RS,7RS,8SR)-tricyclo[5.2.1.0˜2,6˜]dec-4-en-8-yl 2-methylpropanoate, vetyverol, vetyverone, 1-(octahydro-2,3,8,8-tetramethyl-2-naphtalenyl)-1-ethanone (origin: International Flavors and Fragrances, USA), (5RS,9RS,10SR)-2,6,9,10-tetramethyl-1-oxaspiro[4.5]deca-3,6-diene and the (5RS,9SR,10RS) isomer, 6-ethyl-2,10,10-trimethyl-1-oxaspiro[4.5]deca-3,6-diene, 1,2,3,5,6,7-hexahydro-1,1,2,3,3-pentamethyl-4-indenone (origin: International Flavors and Fragrances, USA), a mixture of 3-(3,3-dimethyl-5-indanyl)propanal and 3-(1,1-dimethyl-5-indanyl)propanal (origin: Firmenich SA, Geneva, Switzerland), 3′,4-dimethyl-tricyclo[6.2.1.0(2,7)]undec-4-ene-9-spiro-2′-oxirane (origin: Firmenich SA, Geneva, Switzerland), 9/10-ethyldiene-3-oxatricyclo[6.2.1.0(2,7)]undecane, (perhydro-5,5,8A-trimethyl-2-naphthalenyl acetate (origin: Firmenich SA, Geneva, Switzerland), octalynol, (dodecahydro-3a,6,6,9a-tetramethyl-naphtho[2,1-b]furan, origin: Firmenich SA, Geneva, Switzerland), tricyclo[5.2.1.0(2,6)]dec-3-en-8-yl acetate and tricyclo[5.2.1.0(2,6)]dec-4-en-8-yl acetate as well as tricyclo[5.2.1.0(2,6)]dec-3-en-8-yl propanoate and tricyclo[5.2.1.0(2,6)]dec-4-en-8-yl propanoate, (+)-(1S,2S,3S)-2,6,6-trimethyl-bicyclo[3.1.1]heptane-3-spiro-2′-cyclohexen-4′-one;
Group 5: camphor, borneol, isobornyl acetate, 8-isopropyl-6-methyl-bicyclo[2.2.2]oct-5-ene-2-carbaldehyde, pinene, camphene, 8-methoxycedrane, (8-methoxy-2,6,6,8-tetramethyl-tricyclo[5.3.1.0(1,5)]undecane (origin: Firmenich SA, Geneva, Switzerland), cedrene, cedrenol, cedrol, mixture of 9-ethylidene-3-oxatricyclo[6.2.1.0(2,7)]undecan-4-one and 10-ethylidene-3-oxatricyclo[6.2.1.0(2,7)]undecan-4-one (origin: Firmenich SA, Geneva, Switzerland), 3-methoxy-7,7-dimethyl-10-methylene-bicyclo[4.3.1]decane (origin: Firmenich SA, Geneva, Switzerland);
Group 6: (trimethyl-13-oxabicyclo-[10.1.0]-trideca-4,8-diene (origin: Firmenich SA, Geneva, Switzerland), Ambrettolide LG ((E)-9-hexadecen-16-olide, origin: Firmenich SA, Geneva, Switzerland), pentadecenolide (origin: Firmenich SA, Geneva, Switzerland), muscenone (3-methyl-(4/5)-cyclopentadecenone, origin: Firmenich SA, Geneva, Switzerland), 3-methylcyclopentadecanone (origin: Firmenich SA, Geneva, Switzerland), pentadecanolide (origin: Firmenich SA, Geneva, Switzerland), cyclopentadecanone (origin: Firmenich SA, Geneva, Switzerland), 1-ethoxyethoxy)cyclododecane (origin: Firmenich SA, Geneva, Switzerland), 1,4-dioxacycloheptadecane-5,17-dione, 4,8-cyclododecadien-1-one;
Group 7: (+−)-2-methyl-3-[4-(2-methyl-2-propanyl)phenyl]propanal (origin: Givaudan SA, Vernier, Switzerland), 2,2,2-trichloro-1-phenylethyl acetate.

Preferably, the perfume comprises at least 30%, preferably at least 50%, more preferably at least 60% of ingredients selected from Groups 1 to 7, as defined above. More preferably said perfume comprises at least 30%, preferably at least 50% of ingredients from Groups 3 to 7, as defined above. Most preferably said perfume comprises at least 30%, preferably at least 50% of ingredients from Groups 3, 4, 6 or 7, as defined above.

According to another preferred embodiment, the perfume comprises at least 30%, preferably at least 50%, more preferably at least 60% of ingredients having a log P above 3, preferably above 3.5 and even more preferably above 3.75.

Preferably, the perfume used in the invention contains less than 10% of its own weight of primary alcohols, less than 15% of its own weight of secondary alcohols and less than 20% of its own weight of tertiary alcohols. Advantageously, the perfume used in the invention does not contain any primary alcohols and contains less than 15% of secondary and tertiary alcohols.

According to an embodiment, the oil phase (or the oil-based core) comprises:
25-100 wt % of a perfume oil comprising at least 15 wt % of high impact perfume raw materials having a Log T<−4, and
0-75 wt % of a density balancing material having a density greater than 1.07 g/cm³. The nature of high impact perfume raw materials having a Log T<−4 and density balancing material having a density greater than 1.07 g/cm³ are described in WO2018115250, the content of which are included by reference.

The term “biocide” refers to a chemical substance capable of killing living organisms (e.g. microorganisms) or reducing or preventing their growth and/or accumulation. Biocides are commonly used in medicine, agriculture, forestry, and in industry where they prevent the fouling of, for example, water, agricultural products including seed, and oil pipelines. A biocide can be a pesticide, including a fungicide, herbicide, insecticide, algicide, molluscicide, miticide and rodenticide; and/or an antimicrobial such as a germicide, antibiotic, antibacterial, antiviral, antifungal, antiprotozoal and/or antiparasite.

As used herein, a “pest control agent” indicates a substance that serves to repel or attract pests, to decrease, inhibit or promote their growth, development or their activity. Pests refer to any living organism, whether animal, plant or fungus, which is invasive or troublesome to plants or animals, pests include insects notably arthropods, mites, spiders, fungi, weeds, bacteria and other microorganisms.

According to a particular embodiment, the hydrophobic material is free of any active ingredient (such as perfume). According to this particular embodiment, it comprises, preferably consists of hydrophobic solvents, preferably chosen in the group consisting of isopropyl myristate, tryglycerides (e.g. Neobee® MCT oil, vegetable oils), D-limonene, silicone oil, mineral oil, and mixtures thereof with optionally hydrophilic solvents preferably chosen in the group consisting of 1,4-butanediol, benzyl alcohol, triethyl citrate, triacetin, benzyl acetate, ethyl acetate, propylene glycol (1,2-propanediol), 1,3-propanediol, dipropylene glycol, glycerol, glycol ethers and mixtures thereof.

According to any one of the invention's embodiments, the hydrophobic material represents between 10% and 60% w/w, or even between 15% and 45% w/w, by weight, relative to the total weight of the dispersion as obtained after step b).

According to a particular embodiment, the oil phase essentially consists of the acyl chloride, a polyol and a perfume or flavor oil.

In another step of the process according to the invention, the oil phase of step a) is dispersed into an aqueous solution comprising optionally an amino compound A and/or a base to form an oil-in-water emulsion.

The mean droplet size of the emulsion is preferably comprised between 1 and 1000 microns, more preferably between 1 and 500 microns, and even more preferably between 5 and 50 microns.

By “amino compound A” it should be understood a compound able to react with acyl chloride to form an amide bond.

When present, the amino compound A may be chosen in the group consisting of L-Lysine, L-Lysine ethyl ester, guanidine carbonate, chitosan, 3-aminopropyltriethoxysilane, and mixtures thereof and, when present, the base is preferably NaOH. According to a particular embodiment, the amino compound A is L-Lysine.

When present, the amino compound A or the base is preferably added in an amount comprised between 0.1% and 10% by weight based on the dispersion obtained after step b), more preferably between 0.5% and 5% by weight.

According to the invention, a stabilizer is added in the water phase and/or the oil phase to stabilize the emulsion.

Said stabilizer can be an ionic or non-ionic emulsifier or a colloidal stabilizer.

The stabilizer can be a molecular emulsifier (standard emulsion) or solid particle emulsifier (Pickering emulsion).

“Stabilizer” and “emulsifier” are used indifferently in the present invention.

According to an embodiment, the stabilizer is chosen in the group consisting of gum Arabic, modified starch, polyvinyl alcohol, PVP (polyvinylpyrrolidone), CMC (carboxymethylcellulose), anionic polysaccharides, acrylamide copolymer, inorganic particles, proteins and mixtures thereof

According to an embodiment, the stabilizer is chosen in the group consisting of gum Arabic, modified starch, polyvinyl alcohol, PVP, CMC, anionic polysaccharides, acrylamide copolymer, inorganic particles, protein such as soy protein, rice protein, whey protein, white egg albumin, sodium caseinate, gelatin, bovine serum albumin, hydrolyzed soy protein, hydrolyzed sericin, Pseudocollagen, Silk protein, sericin powder, and mixtures thereof.

When the stabilizer is added in the oil phase, it is preferably chosen in the group consisting of protein such as soy protein, rice protein, whey protein, white egg albumin, sodium caseinate, gelatin, bovine serum albumin, hydrolyzed soy protein, hydrolyzed sericin, Pseudocollagen, Silk protein, sericin powder, and mixtures thereof.

When added in the oil phase, the stabilizer can be pre-dispersed in an inert solvent such as benzyl benzoate.

When the stabilizer is added in the water phase, it is preferably chosen in the group consisting of gum Arabic, modified starch, polyvinyl alcohol, PVP, CMC, anionic polysaccharides, acrylamide copolymer, inorganic particles, protein such as soy protein, rice protein, whey protein, white egg albumin, sodium caseinate, gelatin, bovine serum albumin, hydrolyzed soy protein, hydrolyzed sericin, Pseudocollagen, Silk protein, sericin powder, and mixtures thereof.

According to any one of the above embodiments of the present invention, the dispersion comprises between about 0.01% and 3.0% of at least stabilizer, percentage being expressed on a w/w basis relative to the total weight of the dispersion as obtained after step b). In still another aspect of the invention, the dispersion comprises between about 0.05% and 1.0% of at least a stabilizer. In still another aspect of the invention, the dispersion comprises between about 0.1% and 0.8% of at least a stabilizer.

In another step of the process according to the invention, an amino compound B is added to the oil-in-water emulsion obtained in step b).

By “amino compound B” it should be understood a compound able to react with acyl chloride to form an amide bond.

As non-limiting examples, the amino compound B is chosen in the group consisting of a xylylene diamine, 1,2-diaminocyclohexane, 1,4-diaminocyclohexane, L-lysine, L-Lysine ethyl ester, Jeffamine® (O,O′-Bis(2-aminopropyl) polypropylene glycol-block-polyethylene glycol-block-polypropylene glycol), ethylene diamine, diethylene triamine, spermine, spermidine, polyamidoamine (PAMAM), guanidine carbonate, chitosan, tris-(2-aminoethyl)amine, 3-aminopropyltriethoxysilane, L-arginine, an amine having a disulfide bond such as cystamine, cystamine hydrochloride, cystine, cystine hydrochloride, cystine dialkyl ester, cystine dialkyl ester hydrochloride and mixtures thereof.

According to an embodiment, the amino-compound B is an amine having a disulfide bond and is chosen in the group consisting of cystamine, cystamine hydrochloride, cystine, cystine hydrochloride, cystine dialkyl ester, cystine dialkyl ester hydrochloride and mixtures thereof.

According to another embodiment, the amino-compound B is chosen in the group consisting of xylylene diamine, 1,2-diaminocyclohexane, 1,4-diaminocyclohexane, L-lysine, L-Lysine ethyl ester, Jeffamine® (O,O′-Bis(2-aminopropyl) polypropylene glycol-block-polyethylene glycol-block-polypropylene glycol), ethylene diamine, diethylene triamine, spermine, spermidine, polyamidoamine (PAMAM), guanidine carbonate, chitosan, tris-(2-aminoethyl)amine, 3-aminopropyltriethoxysilane, L-arginine or mixtures thereof.

According to a particular embodiment, the amino compound A and the amino compound B are the same.

According to another particular embodiment, the amino compound A and the amino compound B are different.

According to an embodiment, the weight ratio between the amino compound A and the amino compound B is comprised between 0.5 and 25, preferably between 1.3 and 7.

The process of the invention is notably characterized by the fact that a polyol and an acyl chloride reacts with at least one amino compound during the process. Indeed, without being bound by any theory, the inventors observed that this combination led to stable microcapsules in consumer goods.

The amount of the amino compound B used is typically adjusted so that the molar ratio between the functional groups NH₂ of the amino compound B and COCl of the acyl chloride is comprised between 0.01 and 7.5, preferably from 0.1 to 3.0.

According to an embodiment, a base is added to adjust the pH. One may cite as non-limiting examples guanidine carbonate, sodium bicarbonate or triethanolamine.

Base is preferably added in an amount comprised between 0.1% and 10% by weight based on the dispersion, more preferably between 0.5% and 5% by weight.

This is followed by a curing step d) which allows to end up with microcapsules in the form of a slurry or liquid dispersion. To enhance the kinetics, said step can be performed at a temperature comprised between 50 and 130° C., possibly under pressure, for 15 minutes to 8 hours.

No specific action is required to induce the polymerisation between the acyl chloride and the amino compound(s).

Optional outer coating: According to a particular embodiment of the invention, at the end of step d) or during step d) one may also add to the invention's slurry a polymer selected from the group consisting of a non-ionic polysaccharide, a cationic polymer and mixtures thereof to form an outer coating to the microcapsule.

Non-ionic polysaccharide polymers are well known to a person skilled in the art and are described for instance in WO2012/007438 page 29, lines 1 to 25 and in WO2013/026657 page 2, lines 12 to 19 and page 4, lines 3 to 12. Preferred non-ionic polysaccharides are selected from the group consisting of locust bean gum, xyloglucan, guar gum, hydroxypropyl guar, hydroxypropyl cellulose and hydroxypropyl methyl cellulose.

Cationic polymers are well known to a person skilled in the art. Preferred cationic polymers have cationic charge densities of at least 0.5 meq/g, more preferably at least about 1.5 meq/g, but also preferably less than about 7 meq/g, more preferably less than about 6.2 meq/g. The cationic charge density of the cationic polymers may be determined by the Kjeldahl method as described in the US Pharmacopoeia under chemical tests for Nitrogen determination. The preferred cationic polymers are chosen from those that contain units comprising primary, secondary, tertiary and/or quaternary amine groups that can either form part of the main polymer chain or can be borne by a side substituent directly connected thereto. The weight average (Mw) molecular weight of the cationic polymer is preferably between 10,000 and 3.5M Dalton, more preferably between 50,000 and 1.5M Dalton. According to a particular embodiment, one will use cationic polymers based on acrylamide, methacrylamide, N-vinylpyrrolidone, quaternized N,N-dimethylaminomethacrylate, diallyldimethylammonium chloride, quaternized vinylimidazole (3-methyl-1-vinyl-1H-imidazol-3-ium chloride), vinylpyrrolidone, acrylamidopropyltrimonium chloride, cassia hydroxypropyltrimonium chloride, guar hydroxypropyltrimonium chloride or polygalactomannan 2-hydroxypropyltrimethylammonium chloride ether, starch hydroxypropyltrimonium chloride and cellulose hydroxypropyltrimonium chloride. Preferably copolymers shall be selected from the group consisting of polyquaternium-5, polyquaternium-6, polyquaternium-7, polyquaternium10, polyquaternium-11, polyquaternium-16, polyquaternium-22, polyquaternium-28, polyquaternium-43, polyquaternium-44, polyquaternium-46, cassia hydroxypropyltrimonium chloride, guar hydroxypropyltrimonium chloride or polygalactomannan 2-hydroxypropyltrimethylammonium chloride ether, starch hydroxypropyltrimonium chloride and cellulose hydroxypropyltrimonium chloride. As specific examples of commercially available products, one may cite Salcare® SC60 (cationic copolymer of acrylamidopropyltrimonium chloride and acrylamide, origin: BASF) or Luviquat®, such as the PQ 11N, FC 550 or Style (polyquaternium-11 to 68 or quaternized copolymers of vinylpyrrolidone origin: BASF), or also the Jaguar® (C13S or C17, origin Rhodia).

According to any one of the above embodiments of the invention, there is added an amount of polymer described above comprised between about 0% and 5% w/w, or even between about 0.1% and 2% w/w, percentage being expressed on a w/w basis relative to the total weight of the slurry as obtained after step d). It is clearly understood by a person skilled in the art that only part of said added polymers will be incorporated into/deposited on the microcapsule shell.

Another object of the invention is a process for preparing a microcapsule powder comprising the steps as defined above and an additional step e) consisting of submitting the slurry obtained in step d) to a drying, like spray-drying, to provide the microcapsules as such, i.e. in a powdery form. It is understood that any standard method known by a person skilled in the art to perform such drying is also applicable. In particular the slurry may be spray-dried preferably in the presence of a polymeric carrier material such as polyvinyl acetate, polyvinyl alcohol, dextrins, natural or modified starch, vegetable gums, pectins, xanthans, alginates, carragenans or cellulose derivatives to provide microcapsules in a powder form.

However, one may cite also other drying method such as the extrusion, plating, spray granulation, the fluidized bed, or even a drying at room temperature using materials (carrier, desiccant) that meet specific criteria as disclosed in WO2017/134179.

According to a particular embodiment, the carrier material contains free perfume oil which can be the same or different from the perfume from the core of the microcapsules.

### Multiple Capsule System

According to an embodiment, the microcapsules of the invention (first type of microcapsule) can be used in combination with a second type of microcapsules.

Another object of the invention is a microcapsule delivery system comprising:
the microcapsules of the present invention as a first type of microcapsules, and
a second type of microcapsules, wherein the first type of microcapsules and the second type of microcapsules differ in their hydrophobic material and/or their wall material and/or in their coating material.

### Poly(Amide-Ester) Microcapsule

Another object of the invention is a poly(amide-ester) microcapsule slurry obtainable by the process as described above.

The specific composition of the poly(amide-ester) wall is key in obtaining microcapsules that show the desired stability in the product base (e.g. counteracts efficiently the extraction of the perfume by the surfactants of the consumer product).

Thus, another object of the invention is a poly(amide-ester) core-shell microcapsule or a poly(amide-ester) core-shell microcapsule slurry having at least one poly(amide-ester) core-shell microcapsule, said poly(amide-ester) core-shell microcapsule comprising:
an oil-based core comprising a hydrophobic material, preferably a perfume, and
a poly(amide-ester) shell.

According to an embodiment, the poly(amide-ester) shell comprising:
optionally a stabilizer, preferably from 0% to 75% w/w of a stabilizer,
an acyl chloride, preferably from 10% to 90% w/w, more preferably from 23 to 90% of an acyl chloride,
optionally an amino compound A, preferably from 0 to 50%, more preferably from 5% and 50% w/w of an amino compound A,
an amino compound B, preferably from 1% to 40% w/w of an amino compound B, and
a polyol, preferably from 1 to 60% w/w of a polyol, based on the total weight of the shell.

It should be understood that the sum of all components is 100%.

By “a poly(amide-ester) shell comprising an acyl chloride, at least one amino-compound and a polyol”, it should be understood that the poly(amide-ester) shell is derived from an acyl chloride, at least one amino-compound and a polyol. In other words, it should be understood that the poly(amide-ester) shell comprises the reaction product of an acyl chloride with at least one amino-compound and a polyol.

According to an embodiment, when a stabilizer is present in the shell, it should be understood that the poly(amide-ester) shell is derived from an acyl chloride, at least one amino-compound, a polyol and a stabilizer. In other words, according to this embodiment, it should be understood that the poly(amide-ester) shell comprises the reaction product of an acyl chloride with at least one amino-compound and a stabilizer and a polyol.

Another object of the invention is a solid particle comprising:
a carrier material, preferably a polymeric carrier material chosen in the group consisting of polyvinyl acetate, polyvinyl alcohol, dextrins, natural or modified starch, vegetable gums, pectins, xanthans, alginates, carragenans, cellulose derivatives and mixtures thereof, and
microcapsules as defined above entrapped in said carrier material, and
optionally free perfume entrapped in said carrier material.

Solid particle as defined above and microcapsule powder can be used indifferently in the present invention.

The embodiments described previously regarding the nature of the hydrophobic material, the polyol, the stabilizer, the acyl chloride, the amino compound A and the amino compound B also apply for the poly(amide-ester) microcapsules.

According to a particular embodiment, when the amino compound A is present, the amino compounds A and B comprised in the shell of the poly(amide-ester) microcapsules are different.

The composition of the shell can be quantified for example by elemental analysis and identified by solid-state NMR which are two well-known techniques for the person skilled in the art.

### Perfuming Composition/Consumer Products

The microcapsules of the invention can be used in combination with active ingredients. An object of the invention is therefore a composition comprising:

(i) microcapsules or microcapsule slurry as defined above;

(ii) an active ingredient, preferably chosen in the group consisting of a cosmetic ingredient, skin caring ingredient, perfume ingredient, flavor ingredient, malodour counteracting ingredient, bactericide ingredient, fungicide ingredient, pharmaceutical or agrochemical ingredient, a sanitizing ingredient, an insect repellent or attractant, and mixtures thereof.

The microcapsules of the invention can be used for the preparation of perfuming or flavouring compositions which are also an object of the invention.

The capsules of the invention show very good performance in terms of stability in challenging medium.

Another object of the present invention is a perfuming composition comprising:

(i) microcapsules or microcapsule slurry as defined above, wherein the oil comprises a perfume;

(ii) at least one ingredient selected from the group consisting of a perfumery carrier, a perfumery co-ingredient and mixtures thereof;

(iii) optionally at least one perfumery adjuvant.

As liquid perfumery carrier one may cite, as non-limiting examples, an emulsifying system, i.e. a solvent and a surfactant system, or a solvent commonly used in perfumery. A detailed description of the nature and type of solvents commonly used in perfumery cannot be exhaustive. However, one can cite as non-limiting examples solvents such as dipropyleneglycol, diethyl phthalate, isopropyl myristate, benzyl benzoate, 2-(2-ethoxyethoxy)-1-ethanol or ethyl citrate, which are the most commonly used. For the compositions which comprise both a perfumery carrier and a perfumery co-ingredient, other suitable perfumery carriers than those previously specified, can be also ethanol, water/ethanol mixtures, limonene or other terpenes, isoparaffins such as those known under the trademark Isopar® (origin: Exxon Chemical) or glycol ethers and glycol ether esters such as those known under the trademark Dowanol® (origin: Dow Chemical Company). By “perfumery co-ingredient” it is meant here a compound, which is used in a perfuming preparation or a composition to impart a hedonic effect and which is not a microcapsule as defined above. In other words such a co-ingredient, to be considered as being a perfuming one, must be recognized by a person skilled in the art as being able to at least impart or modify in a positive or pleasant way the odor of a composition, and not just as having an odor.

The nature and type of the perfuming co-ingredients present in the perfuming composition do not warrant a more detailed description here, which in any case would not be exhaustive, the skilled person being able to select them on the basis of his general knowledge and according to the intended use or application and the desired organoleptic effect. In general terms, these perfuming co-ingredients belong to chemical classes as varied as alcohols, lactones, aldehydes, ketones, esters, ethers, acetates, nitriles, terpenoids, nitrogenous or sulphurous heterocyclic compounds and essential oils, and said perfuming co-ingredients can be of natural or synthetic origin. Many of these co-ingredients are in any case listed in reference texts such as the book by S. Arctander, Perfume and Flavor Chemicals, 1969, Montclair, N.J., USA, or its more recent versions, or in other works of a similar nature, as well as in the abundant patent literature in the field of perfumery. It is also understood that said co-ingredients may also be compounds known to release in a controlled manner various types of perfuming compounds.

By “perfumery adjuvant” we mean here an ingredient capable of imparting additional added benefit such as a color, a particular light resistance, chemical stability, etc. A detailed description of the nature and type of adjuvant commonly used in perfuming bases cannot be exhaustive, but it has to be mentioned that said ingredients are well known to a person skilled in the art.

Preferably, the perfuming composition according to the invention comprises between 0.01 and 30% by weight of microcapsules as defined above.

The invention's microcapsules can advantageously be used in many application fields and used in consumer products. Microcapsules can be used in liquid form applicable to liquid consumer products as well as in powder form, applicable to powder consumer products.

According to a particular embodiment, the consumer product as defined above is liquid and comprises:
a) from 2 to 65% by weight, relative to the total weight of the consumer product, of at least one surfactant;
b) water or a water-miscible hydrophilic organic solvent; and
c) microcapsule slurry or microcapsules as defined above,
d) optionally non-encapsulated perfume.

According to a particular embodiment, the consumer product as defined above is in a powder form and comprises:
a) from 2 to 65% by weight, relative to the total weight of the consumer product, of at least one surfactant;
b) microcapsule powder as defined above.
c) optionally perfume powder that is different from the microcapsules defined above.

In the case of microcapsules including a perfume oil-based core, the products of the invention, can in particular be of used in perfumed consumer products such as product belonging to fine fragrance or “functional” perfumery. Functional perfumery includes in particular personal-care products including hair-care, body cleansing, skin care, hygiene-care as well as home-care products including laundry care and air care. Consequently, another object of the present invention consists of a perfumed consumer product comprising as a perfuming ingredient, the microcapsules defined above or a perfuming composition as defined above. The perfume element of said consumer product can be a combination of perfume microcapsules as defined above and free or non-encapsulated perfume, as well as other types of perfume microcapsule than those here-disclosed.

In particular a liquid consumer product comprising:
a) from 2 to 65% by weight, relative to the total weight of the consumer product, of at least one surfactant;
b) water or a water-miscible hydrophilic organic solvent; and
c) a perfuming composition as defined above is another object of the invention.

Also a powder consumer product comprising

(a) from 2 to 65% by weight, relative to the total weight of the consumer product, of at least one surfactant; and
(b) a perfuming composition as defined above is part of the invention.

The invention's microcapsules can therefore be added as such or as part of an invention's perfuming composition in a perfumed consumer product.

For the sake of clarity, it has to be mentioned that, by “perfumed consumer product” it is meant a consumer product which is expected to deliver among different benefits a perfuming effect to the surface to which it is applied (e.g. skin, hair, textile, paper, or home surface) or in the air (air-freshener, deodorizer etc). In other words, a perfumed consumer product according to the invention is a manufactured product which comprises a functional formulation also referred to as “base”, together with benefit agents, among which an effective amount of microcapsules according to the invention.

The nature and type of the other constituents of the perfumed consumer product do not warrant a more detailed description here, which in any case would not be exhaustive, the skilled person being able to select them on the basis of his general knowledge and according to the nature and the desired effect of said product. Base formulations of consumer products in which the microcapsules of the invention can be incorporated can be found in the abundant literature relative to such products. These formulations do not warrant a detailed description here which would in any case not be exhaustive. The person skilled in the art of formulating such consumer products is perfectly able to select the suitable components on the basis of his general knowledge and of the available literature.

Non-limiting examples of suitable perfumed consumer product can be a perfume, such as a fine perfume, a cologne, an after-shave lotion, a body-splash; a fabric care product, such as a liquid or solid detergent, tablets and pods, a fabric softener, a dryer sheet, a fabric refresher, an ironing water, or a bleach; a personal-care product, such as a hair-care product (e.g. a shampoo, hair conditioner, a colouring preparation or a hair spray), a cosmetic preparation (e.g. a vanishing cream, body lotion or a deodorant or antiperspirant), or a skin-care product (e.g. a perfumed soap, shower or bath mousse, body wash, oil or gel, bath salts, or a hygiene product); an air care product, such as an air freshener or a “ready to use” powdered air freshener; or a home care product, such all-purpose cleaners, liquid or power or tablet dishwashing products, toilet cleaners or products for cleaning various surfaces, for example sprays & wipes intended for the treatment/refreshment of textiles or hard surfaces (floors, tiles, stone-floors etc.); a hygiene product such as sanitary napkins, diapers, toilet paper.

Another object of the invention is a consumer product comprising:
a personal care active base, and
microcapsules or microcapsule slurry as defined above or the perfuming composition as defined above,
   wherein the consumer product is in the form of a personal care composition.

Personal care active base in which the microcapsules of the invention can be incorporated can be found in the abundant literature relative to such products. These formulations do not warrant a detailed description here which would in any case not be exhaustive. The person skilled in the art of formulating such consumer products is perfectly able to select the suitable components on the basis of his general knowledge and of the available literature.

The personal care composition is preferably chosen in the group consisting of a hair-care product (e.g. a shampoo, hair conditioner, a colouring preparation or a hair spray), a cosmetic preparation (e.g. a vanishing cream, body lotion or a deodorant or antiperspirant), or a skin-care product (e.g. a perfumed soap, shower or bath mousse, body wash, oil or gel, bath salts, or a hygiene product); Another object of the invention is a consumer product comprising:
a home care or a fabric care active base, and
microcapsules or microcapsule slurry as defined above or the perfuming composition as defined above,
   wherein the consumer product is in the form of a home care or a fabric care composition.

Home care or fabric care active base in which the microcapsules of the invention can be incorporated can be found in the abundant literature relative to such products. These formulations do not warrant a detailed description here which would in any case not be exhaustive. The person skilled in the art of formulating such consumer products is perfectly able to select the suitable components on the basis of his general knowledge and of the available literature.

Preferably, the consumer product comprises from 0.1 to 15 wt %, more preferably between 0.2 and 5 wt % of the microcapsules of the present invention, these percentages being defined by weight relative to the total weight of the consumer product. Of course the above concentrations may be adapted according to the benefit effect desired in each product.

### Fabric Softener

An object of the invention is a consumer product in the form of a fabric softener composition comprising:
a fabric softener active base; preferably chosen in the group consisting of dialkyl quaternary ammonium salts, dialkyl ester quaternary ammonium salts (esterquats), Hamburg esterquat (HEQ), TEAQ (triethanolamine quat), silicones and mixtures thereof, preferably in an amount comprised between 85 and 99.95% by weight based on the total weight of the composition,
a microcapsule slurry or microcapsules as defined above, preferably in an amount comprised between 0.05 to 15 wt %, more preferably between 0.1 and 5 wt % by weight based on the total weight of the composition.

### Liquid Detergent

An object of the invention is a consumer product in the form of a liquid detergent composition comprising:
a liquid detergent active base; preferably chosen in the group consisting of anionic surfactant such as alkylbenzenesulfonate (ABS), secondary alkyl sulfonate (SAS), primary alcohol sulfate (PAS), lauryl ether sulfate (LES), methyl ester sulfonate (MES) and nonionic surfactant such as alkyl amines, alkanolamide, fatty alcohol poly(ethylene glycol) ether, fatty alcohol ethoxylate (FAE), ethylene oxide (EO) and propylene oxide (PO) copolymers, amine oxydes, alkyl polyglucosides, alkyl polyglucosamides, preferably in an amount comprised between 85 and 99.95% by weight based on the total weight of the composition,
a microcapsule slurry or microcapsules as defined above, preferably in an amount comprised between 0.05 to 15 wt %, more preferably between 0.1 and 5 wt % by weight based on the total weight of the composition.

### Solid Detergent

An object of the invention is a consumer product in the form of a solid detergent composition comprising:
a solid detergent active base; preferably chosen in the group consisting of anionic surfactant such as alkylbenzenesulfonate (ABS), secondary alkyl sulfonate (SAS), primary alcohol sulfate (PAS), lauryl ether sulfate (LES), methyl ester sulfonate (MES) and nonionic surfactant such as alkyl amines, alkanolamide, fatty alcohol poly(ethylene glycol) ether, fatty alcohol ethoxylate (FAE), ethylene oxide (EO) and propylene oxide (PO) copolymers, amine oxydes, alkyl polyglucosides, alkyl polyglucosamides, preferably in an amount comprised between 85 and 99.95% by weight based on the total weight of the composition,
a microcapsule slurry or microcapsules as defined above, preferably in an amount comprised between 0.05 to 15 wt %, more preferably between 0.1 and 5 wt % by weight based on the total weight of the composition.

### Shampoo/Shower Gel

An object of the invention is a consumer product in the form of a shampoo or a shower gel composition comprising:
a shampoo or a shower gel active base; preferably chosen in the group consisting of sodium alkylether sulfate, ammonium alkylether sulfates, alkylamphoacetate, cocamidopropyl betaine, cocamide MEA, alkylglucosides and aminoacid based surfactants and mixtures thereof, preferably in an amount comprised between 85 and 99.95% by weight based on the total weight of the composition,
a microcapsule slurry or microcapsules as defined above, preferably in an amount comprised between 0.05 to 15 wt %, more preferably between 0.1 and 5 wt % by weight based on the total weight of the composition.

### Rinse-Off Conditioner

An object of the invention is a consumer product in the form of a rinse-off conditioner composition comprising:
a rinse-off conditioner active base; preferably chosen in the group consisting of cetyltrimonium chloride, stearyl trimonium chloride, benzalkonium chloride, behentrimonium chloride and mixture thereof, preferably in an amount comprised between 85 and 99.95% by weight based on the total weight of the composition,
a microcapsule slurry or microcapsules as defined above, preferably in an amount comprised between 0.05 to 15 wt %, more preferably between 0.1 and 5 wt % by weight based on the total weight of the composition.

### Hair Coloration

An object of the invention is a consumer product in the form of an oxidative hair coloring composition comprising:
an oxidizing phase comprising an oxidizing agent and an alkaline phase comprising an alkakine agent, a dye precursor and a coupling compound; wherein said dye precursor and said coupling compound form an oxidative hair dye in the presence of the oxidizing agent, preferably in an amount comprised between 85 and 99.95% by weight based on the total weight of the composition,
a microcapsule slurry or microcapsules as defined above, preferably in an amount comprised between 0.05 to 15 wt %, more preferably between 0.1 and 5 wt % by weight based on the total weight of the composition.

By “oxidative hair coloring composition”, it is meant a composition comprising two groups of colorless dye molecules: the dye precursor and the coupling agent. Upon reaction with each other through an oxidation process, they form a wide range of colored molecules (dyes) that are then trapped into the hair due their size. In other words, the dye precursor and the coupling compound form an oxidative hair dye in the presence of the oxidizing agent.

“Dye precursor” and “oxidative dye precursor” are used indifferently in the present invention.

Dye precursors can be aromatic compounds derived from benzene substituted by at least two electron donor groups such as NH₂ and OH in para or ortho positions to confer the property of easy oxidation.

According to an embodiment, dye precursors are chosen in the group consisting of p-phenylene diamine, 2,5-diamino toluene, N,N-bis(2-hydroxymethyl)-p-phenylene diamine, 4-aminophenol, 1,4-diamino-benzene, and mixtures thereof.

The primary dye precursors is used in combination with coupling agents. Coupling agents are preferably aromatic compounds derived from benzene and substituted by groups such as NH₂ and OH in the meta position and do not produce color singly, but which modify the color, shade or intensity of the colors developed by the dye precursor.

According to an embodiment, the coupling agent is chosen in the group consisting of resorcinol, 2-methyl resorcinol, 4-chlororesorchinol, 2,5-diamino-toluene, 1,3-diamino-benzene, 2,4-diaminophenoxyethanol HCl, 2-amino-hydroxyethylaminoanisole sulfate, 4-amino-2-hydroxytoluene, and mixtures thereof.

The oxidative dye precursor is preferably used in an amount comprised between 0.001% and 5%, preferably between 0.1% and 4% by weight based on the total weight of the composition.

The use of oxidative dye precursors and coupling agents in hair coloring formulation have been widely disclosed in the prior art and is well-known from the person skilled in the art. One may cite for example EP0946133A1, the content of which is incorporated by reference.

The alkaline phase comprises an alkaline agent, preferably chosen in the group consisting of ammonia hydroxide, ammonia carbonate, ethanolamine, potassium hydroxide, sodium borate, sodium carbonate, triethanolamine and mixtures thereof.

The alkaline agent is preferably used in an amount comprised between 1% and 10%, preferably between 3% and 9% by weight based on the total weight of the composition.

According to the invention, the coupling agent and the dye precursor in an alkaline medium form an oxidative hair dye in the presence of the oxidizing agent.

The oxidizing agent will supply the necessary oxygen gas to develop color molecules and create a change in hair color.

The oxidizing agent should be safe and effective for use in the compositions herein.

Preferably, the oxidizing agents suitable for use herein will be soluble in the compositions according to the present invention when in liquid form and/or in the form intended to be used.

Preferably, oxidizing agents suitable for use herein will be water-soluble. Suitable oxidizing agents for use herein are selected from inorganic peroxygen oxidizing agents, preformed organic peroxyacid oxidizing agents and organic peroxide oxidizing agents or mixtures thereof.

The oxidizing agent is preferably used in an amount comprised between 5 and 30%, preferably between 5 and 25% by weight based on the total weight of the composition.

Components commonly used in cosmetic compositions may be added into the hair coloring composition as defined in the present invention. One may cite for example, surfactants, cationic polymers, oily substances, silicone derivatives, free perfume, preservatives, ultraviolet absorbents, antioxidants, germicides, propellants, thickeners.

According to a particular embodiment, the hair coloring composition comprises one or more quaternary ammonium compounds, preferably chosen in the group consisting of cetyltrimonium chloride, stearyl trimonium chloride, benzalkonium chloride, behentrimonium chloride and mixture thereof to confer hair conditioner benefits.

### Perfuming Composition

According to a particular embodiment, the consumer product is in the form of a perfuming composition comprising:
0.1 to 30%, preferably 0.1 to 20% of microcapsules or microcapsule slurry as defined previously,
0 to 40%, preferably 3-40% of perfume, and
20-90%, preferably 40-90% of ethanol, by weight based on the total weight of the perfuming composition.

The invention will now be further described by way of examples. It will be appreciated that the invention as claimed is not intended to be limited in any way by these examples.

### EXAMPLES

### Materials

TABLE 1
List of ingredients used in the examples

| Ingredients | Function |
|---|---|
| BTC¹⁾ | Acyl chloride |
| mXDA²⁾ | Amino compound |
| L-lysine³⁾ | Amino compound |
| Jeffamine ® D230⁴⁾ | Amino compound |
| EDA⁵⁾ | Amino compound |
| Diethylene triamine⁶⁾ | Amino compound |
| Spermine dihydrate⁷⁾ | Amino compound |
| 1,3-diaminopropane⁸⁾ | Amino compound |
| Guanidine carbonate⁹⁾ | Amino compound |
| Cystamine hydrochloride¹⁰⁾ | Amino compound |
| Gum arabic¹¹⁾ | Colloidal stabilizer |
| Bovin Serum Albumin¹²⁾ | Colloidal stabilizer |
| Triethanolamine¹³⁾ | Polyol |
| Trimethylolpropane¹⁴⁾ | Polyol |

¹⁾1,3,5-benzene tricarbonyl chloride; origin: Aldrich, Switzerland
²⁾m-xylylyene diamine; origin: Aldrich, Switzerland
³⁾L-Lysine; origin: Aldrich, Switzerland
⁴⁾Jeffamine ® D230; origin: Aldrich, Switzerland
⁵⁾Ethylene diamine; origin: Aldrich, Switzerland
⁶⁾Diethylene triamine; origin: Aldrich, Switzerland
⁷⁾Spermine dihydrate; origin: Aldrich, Switzerland
⁸⁾1,3-diaminopropane; origin: Aldrich, Switzerland
⁹⁾Guanidine carbonate; origin: Aldrich, Switzerland
¹⁰⁾Cystamine hydrochloride, origin: Aldrich, Switzerland
¹¹⁾Acacia gum Superstab AA, origin: Nexira, France
¹²⁾Bovin Serum Albumin; origin: Aldrich, Switzerland
¹³⁾Triethanolamine; origin: Aldrich, Switzerland
¹⁴⁾Trimethylolpropane; origin: Aldrich, Switzerland

### Perfume Oil Composition:

TABLE 2
Perfume oil composition

| Raw Materials | % in oil |
|---|---|
| 2,4-Dimethyl-3-cyclohexene-1-carbaldehyde | 3.30% |
| Allyl Heptanoate | 5.50% |
| Allyl amyl glycolate | 10.99% |
| Delta Damascone | 1.65% |
| Verdyl acetate | 20.30% |
| Methyl dihydrojasmonate ¹⁾ | 4.95% |
| 1-(octahydro-2,3,8,8-tetramethyl-2-naphtalenyl)-1-ethanone ²⁾ | 16.49% |
| Ald. Hexylcinnamique | 9.89% |
| Ethyl-2-methylvalerate | 3.3% |
| (+−)-2-methyl-3-[4-(2-methyl-2-propanyl)phenyl]propanal | 21.98% |
| (Z)-hex-3-en-1-yl butyrate | 1.1% |
| (−)-(8R)-8,12-epoxy-13,14,15,16-tetranorlabdane ³⁾ | 0.55% |
| Total | 100% |

¹⁾ Firmenich SA, Geneva, Switzerland
²⁾ International Flavors & Fragrances, USA
³⁾ Firmenich SA, Geneva, Switzerland

### Example 1

### Preparation of Poly(Amide-Ester) Microcapsules with a Colloidal Stabilizer in Oil Phase

### Capsules A: Preparation of Capsules a by Using Trimethylolpropane as a Polyol.

1,3,5-Benzene tricarbonyle chloride (BTC, 0.88 g, Table 1) was dissolved in benzyl benzoate (2 g). Bovin Serum Albumin (0.95 g) was dispersed in benzyl benzoate at 60° C. and the acyl chloride solution was added and stirred for two minutes. Trimethylolpropane (0.45 g) was dissolved in ethyl acetate (2 g) at room temperature and the solution was added to a second solution of 1,3,5-benzene tricarbonyle chloride (0.88 g) in benzyl benzoate (2 g). The solution and the dispersion were added to the perfume oil (25 g, Table 2) at room temperature to form an oil phase. Oil phase was added to an aqueous solution of L-Lysine (2.5 g) (amino compound A) in water (95 g). Reaction mixture was stirred with an Ultra Turrax at 24,000 rpm for one minute to afford an emulsion. Polyamine (Amino compound B) Table 3) was dissolved in water (5 g) and this solution was added dropwise to the emulsion. The reaction mixture was stirred at 30° C. for 4 h to afford a white dispersion.

TABLE 3
Capsule A compositions

| Capsules | Amino-compound B | Amine (g) |
|---|---|---|
| A1 | Ethylene diamine | 0.5 |
| A2 | m-Xylylene diamine | 1.08 |
| A3 | Spermine dihydrate | 1.90 |
| A4 | Diethylene triamine | 0.82 |
| A5 | Cystamine hydrochloride | 1.80 |

### Example 2

### Preparation of Poly(Amide-Ester) Microcapsules with Colloidal Stabilizer in Water Phase

Capsules B— Preparation of Capsules B with Trimethylolpropane as a Polyol

Trimethylolpropane (TMP) was dissolved in ethyl acetate (5 g) at 40° C. Solution of polyol was added on a solution of 1,3,5-benzene tricarbonyle chloride in benzyl benzoate (5 g), the solution was left for stirring for 10 minutes at room temperature. The solution was added to the perfume oil (25 g, Table 2) to form an oil phase. L-Lysine (3.5 g, 24 mmol) was dissolved in an aqueous solution of gum Arabic (95 g, 1 wt %). The solution was stirred for 15 minutes to form an aqueous phase. Oil phase was added to the aqueous phase and the reaction mixture was stirred with an Ultra Turrax at 24,000 rpm for 30 seconds to afford an emulsion. Polyamine (Table 7) was dissolved in water (1 g) and this solution was added dropwise to the emulsion. A solution of guanidine carbonate (10 wt %) was added to obtain a pH value at 7. The reaction mixture was stirred at 200 rpm with an anchor at 30° C. for 2 h, then at 60° C. for 2 h to afford a white dispersion.

TABLE 7
Capsule B compositions

| Cap- | Acid | TMP | Amino | | Amino | |
|---|---|---|---|---|---|---|
| sules | Chloride (g) | (g) | compound A | (g) | compound B | (g) |
| B1 | 1.77 | 0.90 | L-Lysine | 3.5 | 1,2-diamino | 0.14 |
| | | | | | cyclohexane | |
| B2 | 1.77 | 0.90 | L-Lysine | 3.5 | m-xylylene | 0.17 |
| | | | | | diamine | |
| B3 | 1.77 | 0.90 | L-Lysine | 3.5 | 1,4-diamino | 0.14 |
| | | | | | cyclohexane | |
| B4 | 2.66 | 0.90 | L-Lysine + | 2.5 | Jeffamine | 1.18 |
| | | | Guanidine | 10.78 | T403 | |
| | | | Carbonate 30% | | | |
| B5 | 2.66 | 0.45 | L-Lysine + | 2.5 | Jeffamine | 2.35 |
| | | | Guanidine | 10.25 | T403 | |
| | | | Carbonate 30% | | | |
| B6 | 2.66 | 0.90 | L-Lysine + | 2.5 | Lysine ethyl | 0.99 |
| | | | Guanidine | 13.45 | ester | |
| | | | Carbonate 30% | | | |
| B7 | 2.66 | 0.45 | L-Lysine + | 2.5 | Lysine ethyl | 1.95 |
| | | | Guanidine | 12.45 | ester | |
| | | | Carbonate 30% | | | |
| B8 | 2.65 | 0.45 | L-Lysine | 2.5 | Ethylene | 0.48 |
| | | | | | diamine | |
| B9 | 2.65 | 0.45 | L-Lysine | 2.5 | Diethylene | 0.82 |
| | | | | | triamine | |
| B10 | 2.65 | 0.45 | L-Lysine | 2.5 | Spermine | 1.90 |
| B11 | 2.65 | 0.45 | L-Lysine + | 2.5 | Cystamine | 1.80 |
| | | | Guanidine | 6.38 | | |
| | | | Carbonate 30% | | | |
| B12 | 1.77 | 0.90 | L-Lysine | 2.5 | 1,2-diamino | 0.91 |
| | | | | | cyclohexane | |
| B13 | 7.72 | 1.30 | L-Lysine | 7.30 | m-xylylene | 3.60 |
| | | | | | diamine | |

Capsules C— Preparation of Capsules C with Triethanolamine as a Polyol

Capsules C were prepared as described above for capsules B with triethanolamine (0.50 g) in ethyl acetate (2.5 g), 1,3,5-benzene tricarbonyle chloride (1.76 g) in benzyl benzoate (5 g), m-xylylene diamine in water (5 g), L-Lysine (2.5 g) in an aqueous solution of gum Arabic at 1 wt % (95 g), for 4 h at 30° C.

Capsules D—Preparation of Capsules D with Ethylene Glycol as a Polyol

Ethylene glycol (0.12 g) was dissolved in ethyl acetate (2.5 g) at 40° C. Solution of polyol (ethylene glycol) was added on a solution of 1,3,5-benzene tricarbonyle chloride (0.35 g) in benzyl benzoate (2.5 g), the solution was left for stirring for 60 minutes at 60° C. The solution was added to the perfume oil (25 g, Tables 2) in which colloidal stabilizer (Sodium Caseinate 0.95 g) is dispersed and a solution of 1,3,5-benzen tricarbonyle chloride (1.4 g) in benzyl benzoate (2.5 g) to form an oil phase. L-Lysine (2.5 g, 17 mmol) was dissolved water. The solution was stirred for 15 minutes to form an aqueous phase. Oil phase was added to the aqueous phase and the reaction mixture was stirred with an Ultra Turrax at 24,000 rpm for 30 seconds to afford an emulsion. Ethylene diamine (0.2 g) was dissolved in water (5 g) and this solution was added dropwise to the emulsion. The reaction mixture was stirred at 250 rpm in incubator at 30° C. for 4 h.

The average size of microcapsules D is around 30 μm.

Microcapsules show a perfume leakage of 16% in a fabric softener base (see composition in table 5) for 3 days at 43° C.

Capsules E—Preparation of Capsules E with Pentaerythritol as a Polyol

Capsules E were prepared as described above for capsules D with pentaerythritol (0.2 g) in ethanol (2.5 g), 1,3,5-benzene tricarbonyle chloride (1.76 g total) in benzyl benzoate (5 g total), ethylene diamine (0.18 g) in water (5 g), L-Lysine (2.5 g) in water, for 4 h at 30° C.

TGA measurement from 30° C. to 50° C. (5° C./min) and hold at 50° C. for 250 min show that microcapsules retain 100% of perfume oil after 250 min.

Capsules F— Preparation of Capsules F with Di(Trimethylolpropane) as a Polyol

Capsules F were prepared as described above for capsules D with di(trimethylolpropane) (0.25 g) in acetone (2.5 g), 1,3,5-benzene tricarbonyle chloride (1.76 g total) in benzyl benzoate (5 g total), ethylene diamine (0.23 g) in water (5 g), L-Lysine (2.5 g) in water, for 4 h at 30° C. The average size of microcapsules F is around 30 μm.

### Example 3

### Preparation of Poly(Amide-Ester) Microcapsules with Polyphenols

### Capsules G: Preparation of Capsules G by Using Phloroglucinol as a Polyol

1,3,5-Benzene tricarbonyle chloride (BTC, 1.7799 g, Table 1) was dissolved in benzyl benzoate (5.02 g). Sodium Caseinate (0.95 g) was dispersed in 5.10 g benzyl benzoate. The solution and the dispersion were added to the perfume oil (25.30 g, Table 2) at room temperature to form an oil phase. The oil phase was then quickly added to an aqueous solution of L-Lysine (2.5 g) in water (94 g) before emulsifying with an Ultra Turrax at 24,000 rpm for 30-45 seconds. A beforehand-prepared solution of phloroglucinol (0.13 g) in ethanol (1 g) was then added to the emulsion while stirring with a propeller at 400 rpm. Three to five minutes later, a solution of ethylene diamine (EDA, 0.18 g, Table 1) dissolved in water (5 g) was slowly added to the reaction mixture while stirring. The reaction mixture was stirred at 30° C. for 4 h to afford a white dispersion.

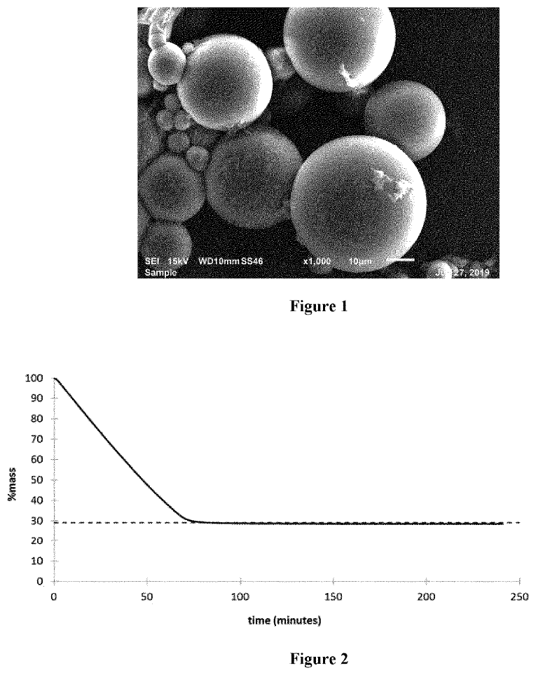

The average size of microcapsule G is 50 microns (see FIG. 1).

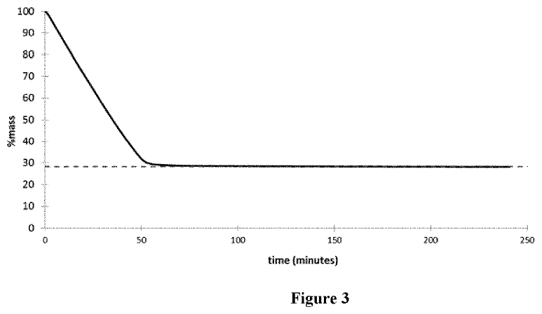

TGA measurement from 30° C. to 50° C. (10° C./min) and hold at 50° C. for 240 min show that microcapsules retain 100% of perfume oil after 240 min (see FIG. 2).

### Stability in Fabric Softener Base of Microcapsule G:

The amount of microcapsule suspension G corresponding to 0.116% perfume was dispersed in a fabric softener (see Table 5) and stored for 3 days at 38° C. Perfume leakage was then quantified and measured at 14% after 3 days.

### Capsules H: Preparation of Capsules H by Using a Condensed Tannin as Polyphenol.

1,3,5-Benzene tricarbonyle chloride (BTC, 1.7878 g, Table 1) was dissolved in benzyl benzoate (5.20 g). Sodium Caseinate (0.96 g) was dispersed in 5.05 g benzyl benzoate. The solution and the dispersion were added to the perfume oil (25.22 g, Table 2) at room temperature to form an oil phase. The oil phase was then quickly added to an aqueous solution of L-Lysine (2.51 g) in water (95 g) before emulsifying with an Ultra Turrax at 24,000 rpm for 30-45 seconds. A beforehand-prepared solution of Tan' activ U condensed tannin (0.20 g) in water (5 g) was then added to the emulsion while stirring with a propeller at 400 rpm. Three to five minutes later, a solution of ethylene diamine (EDA, 0.24 g, Table 1) dissolved in water (5 g) was slowly added to the reaction mixture while stirring. The reaction mixture was stirred at 30° C. for 4 h to afford a white dispersion.

The average size of microcapsule H is 50 microns.

TGA measurement from 30° C. to 50° C. (10° C./min) and hold at 50° C. for 240 min show that microcapsules retain 100% of perfume oil after 240 min (see FIG. 3).

Stability in fabric softener base of microcapsule H:

The amount of microcapsule suspension H corresponding to 0.116% perfume was dispersed in a fabric softener (see composition in Table 5) and stored for 3 days at 38° C. Perfume leakage was then quantified and measured at 14% after 3 days.

### Example 4

### Fabric Softener Composition

Capsules A, B, C, D, E, F, G or H (A-H) are dispersed in a fabric softener base described in table to obtain a concentration of encapsulated perfume oil at 0.22%.

TABLE 5
Fabric Softener composition

| Product | Wt % |
|---|---|
| Stepantex VL 90A | 8.88 |
| Calcium Chloride Sol. 10% | 0.36 |
| Proxel GXL | 0.04 |
| Perfume | 1 |
| Water | 89.72 |
| TOTAL | 100 |

### Example 5

### Liquid Detergent Composition

Capsules A-H are dispersed in a liquid detergent base described in table 6 to obtain a concentration of encapsulated perfume oil at 0.22%.

TABLE 6
Liquid detergent composition

| Ingredients | Concentration [wt %] |
|---|---|
| Sodium C14-17 Alkyl Sec Sulfonate¹⁾ | 7 |
| Fatty acids, C12-18 and C18-unsaturated²⁾ | 7.5 |
| C12/14 fatty alcohol polyglycol ether with 7 | 17 |
| mol EO³⁾ | |
| Triethanolamine | 7.5 |
| Propylene Glycol | 11 |
| Citric acid | 6.5 |
| Potassium Hydroxyde | 9.5 |
| Protease | 0.2 |
| Amylase | 0.2 |
| Mannanase | 0.2 |
| Acrylates/Steareth-20 Methacrylate | 6 |
| structuring Crosspolymer⁴⁾ | |
| Deionized Water | 27.4 |

¹⁾Hostapur SAS 60; Origin: Clariant
²⁾Edenor K 12-18; Origin: Cognis
³⁾Genapol LA 070; Origin: Clariant
⁴⁾Aculyn 88; Origin: Dow Chemical

### Example 6

### Rinse-Off Conditioner

Capsules A-H are dispersed in a rinse-off conditioner base described in table 7 to obtain a concentration of encapsulated perfume oil at 0.5%.

TABLE 7
rinse-off conditioner composition

| | | Concentration |
|---|---|---|
| | Ingredients | [wt %] |
| A | Water deionized | 81.8 |
| | Behentrimonium Chloride ¹⁾ | 2.5 |
| | Hydroxyethylcellulose ²⁾ | 1.5 |
| B | Cetearyl Alcohol ³⁾ | 4 |
| | Glyceryl Stearate (and) PEG-100 Stearate ⁴⁾ | 2 |
| | Behentrimonium Methosulfate (and) Cetyl alcohol | 4 |
| | (and) Butylene Glycol ⁵⁾ | |
| | Ethoxy (20) Stearyl Alcohol ⁶⁾ | 1 |
| C | Amodimethicone (and) Trideceth-12 (and) | 3 |
| | Cetrimonium Chloride ⁷⁾ | |
| | Chlorhexidine Digluconate ⁸⁾ 20% aqueous solution | 0.2 |
| D | Citric acid 10% aqueous sol. till pH 3.5-4 | q.s. |
| | T O T A L: | 100 |

¹⁾ Genamin KDM P, Clariant
²⁾ Tylose H10 Y G4, Shin Etsu
³⁾ Lanette O, BASF
⁴⁾ Arlacel 165-FP-MBAL-PA-(RB), Croda
⁵⁾ Incroquat Behenyl TMS-50-MBAL-PA-(MH) HA4112, Croda
⁶⁾ SP Brij S20 MBAL-PA(RB), Croda
⁷⁾ Xiameter DC MEM-0949 Emulsion, Dow Corning
⁸⁾ Alfa Aesar

### Example 7

### Shampoo Composition

Capsules A-H of the present invention are weighed and mixed in a shampoo composition to add the equivalent of 0.2% perfume.

TABLE 8
Shampoo composition

| | | Concentration |
|---|---|---|
| | Ingredients | [wt %] |
| A | Water deionized | 44.4 |
| | Polyquaternium-10 ¹⁾ | 0.3 |
| | Glycerin 85% ²⁾ | 1 |
| | DMDM Hydantoin ³⁾ | 0.2 |
| B | Sodium Laureth Sulfate ⁴⁾ | 28 |
| | Cocamidopropyl Betaine ⁵⁾ | 3.2 |
| | Disodium Cocoamphodiacetate ⁶⁾ | 4 |
| | Ethoxy (20) Stearyl Alcohol ⁶⁾ | 1 |
| C | Sodium Laureth Sulfate ⁴⁾ | 3 |
| | Glyceryl Laureate ⁷⁾ | 0.2 |
| D | Water deionized | 1 |
| | Sodium Methylparaben ⁸⁾ | 0.1 |
| E | Sodium Chloride 10% aqueous sol. | 15 |
| | Citric acid 10% aqueous sol. till pH 5.5-6 | q.s. |
| | Perfume | 0.5 |
| | T O T A L: | 100 |

¹⁾ Ucare Polymer JR-400, Noveon
²⁾ Schweizerhall
³⁾ Glydant, Lonza
⁴⁾ Texapon NSO IS, Cognis
⁵⁾ Tego Betain F 50, Evonik
⁶⁾ Amphotensid GB 2009, Zschimmer & Schwarz
⁷⁾ Monomuls 90 L-12, Gruenau
⁸⁾ Nipagin Monosodium, NIPA

### Example 8

### Antiperspirant Roll-on Emulsion Composition

Capsules A-H of the present invention are weighed and mixed in antiperspirant roll-on emulsion composition to add the equivalent of 0.2% perfume.

TABLE 9
Antiperspirant roll-on emulsion composition

| Ingredient | Amount (wt %) |
|---|---|
| Steareth-2¹⁾ (Part A) | 3.25 |
| Steareth-21²⁾ (Part A) | 0.75 |
| PPG-15 Stearyl Ether³⁾ (Part A) | 4 |
| WATER deionised (Part B) | 51 |
| Aluminum Chlorohydrate 50% | 40 |
| aqueous solution⁴⁾ (Part C) | |
| Fragrance (Part D) | 1 |

¹⁾BRIJ 72; origin: ICI
²⁾BRIJ 721; origin: ICI
³⁾ARLAMOL E; origin: UNIQEMA-CRODA
⁴⁾LOCRON L; origin: CLARIAN

Part A and B are heated separately to 75° C.; Part A is added to Part B under stirring and the mixture is homogenized for 10 min. Then, the mixture is cooled under stirring; and Part C is slowly added when the mixture reached 45° C. and Part D when the mixture reached at 35° C. while stirring. Then the mixture is cooled to room temperature.

### Example 9

### Shower-Gel Composition

Capsules A-H of the present invention are weighed and mixed in the following composition to add the equivalent of 0.2% perfume.

TABLE 10
Shower gel composition

| | Amount | |
|---|---|---|
| Ingredients | (% wt) | Function |
| WATER deionised | 49.350 | Solvent |
| Tetrasodium EDTA ¹⁾ | 0.050 | Chelating agent |
| Acrylates Copolymer²⁾ | 6.000 | Thickener |
| Sodium C12-C15 Pareth Sulfate ³⁾ | 35.000 | Surfactant |
| Sodium Hydroxide 20% aqueous solution | 1.000 | pH adjuster |
| Cocamidopropyl Betaine⁴⁾ | 8.000 | Surfactant |
| Methylchloroisothiazolinone and | 0.100 | Preservative |
| Methylisothiazolinone⁵⁾ | | |
| Citric Acid (40%) | 0.500 | pH adjuster |

¹⁾ EDETA B POWDER; trademark and origin: BASF
²⁾CARBOPOL AQUA SF-1 POLYMER; trademark and origin: NOVEON
³⁾ ZETESOL AO 328 U; trademark and origin: ZSCHIMMER & SCHWARZ
⁴⁾TEGO-BETAIN F 50; trademark and origin: GOLDSCHMIDT
⁵⁾KATHON CG; trademark and origin: ROHM & HASS

1. A process for preparing a core-shell poly(amide-ester) microcapsule slurry comprising the following steps:
a) Dissolving at least one acyl chloride in a hydrophobic material to form an oil phase;
b) Dispersing the oil phase obtained in step a) into a water phase comprising optionally an amino compound A or a base to form an oil-in-water emulsion;
c) Adding to the oil-in-water emulsion obtained in step b) an amino compound B; and
d) Performing a curing step to form poly(amide-ester) microcapsule in the form of a slurry,
wherein a stabilizer is added in step a) and/or in step b), and
wherein a polyol is added in step a) and/or in step b) and/or in step c).

2. The process according to claim 1, wherein the acyl chloride is selected from the group consisting of 1,3,5-benzene tricarbonyl chloride, benzene-1,2,4-tricarbonyl trichloride, benzene-1,2,4,5-tetracarbonyl tetrachloride, cyclohexane-1,3,5-tricarbonyl trichloride, isophthalyol dichloride, diglycolyl dichloride, terphthaloyl chloride, succinic dichloride, and mixtures thereof.

3. The process according to claim 1, wherein the water phase comprises the amino compound A.

4. The process according to claim 1, wherein the amino compound A is selected from the group consisting of L-Lysine, L-Lysine ethyl ester, guanidine carbonate, chitosan, 3-aminopropyltriethoxysilane, and mixtures thereof.

5. The process according to claim 1, wherein the amino compound B is selected from the group consisting of cystamine, cystamine hydrochloride, cystine, cystine hydrochloride, cystine dialkyl ester, cystine dialkyl ester hydrochloride, a xylylene diamine, 1,2-diaminocyclohexane, 1,4-diaminocyclohexane, L-lysine, L-Lysine ethyl ester, (O,O′-Bis(2-aminopropyl) polypropylene glycol-block-polyethylene glycol-block-polypropylene glycol, ethylene diamine, diethylene triamine, spermine, spermidine, polyamidoamine (PAMAM), guanidine carbonate, chitosan, tris-(2-aminoethyl)amine, 3-aminopropyltriethoxysilane, L-arginine, and mixtures thereof.

6. The process according to claim 3, wherein the amino-compound A and the amino-compound B are different.

7. The process according to claim 1, wherein the stabilizer is selected from the group consisting of gum Arabic, modified starch, polyvinyl alcohol, polyvinylpyrrolidone, carboxymethylcellulose, anionic polysaccharides, acrylamide copolymer, inorganic particles, proteins, and mixtures thereof.

8. The process according to claim 1, wherein the molar ratio between functional groups NH₂ of the amino compound B and functional groups COCl of the acyl chloride is comprised between 0.01 and 7.5.

9. The process according to claim 1, wherein the weight ratio between acyl chloride and the hydrophobic material is comprised between 0.02 and 0.09.

10. The process according to claim 1, wherein the polyol is selected from the group consisting of triethanolamine, di(trimethylolpropane), ethylene glycol, glycerol, 1,4-butanediol, 1,2-hexanediol, 1,6-hexanediol, 2-ethyl-2-(hydroxymethyl)propane-1,3-diol (trimethylolpropane, TMP), 2,2-bis(hydroxymethyl)propane-1,3-diol (pentaerythritol), 2-amino-2-ethylpropane-1,3-diol, 2-amino-2-(hydroxymethyl)propane-1,3-diol, 2,2′-azanediylbis(ethan-1-ol), 2-aminopropane-1,3-diol, 2-amino-2-methylpropane-1,3-diol, polyphenols, and mixtures thereof.

11. A poly(amide-ester) core-shell microcapsule comprising:
an oil-based core comprising a hydrophobic material, and
a poly(amide-ester) shell.

12. The poly(amide-ester) core-shell microcapsule according to claim 11, wherein the poly(amide-ester) shell comprises:
optionally a stabilizer,
an acyl chloride,
optionally an amino compound A,
an amino compound B, and
a polyol.

13. A perfuming composition comprising:
(i) Perfume microcapsule as defined in claim 11, wherein the hydrophobic material comprises a perfume,
(ii) At least one ingredient selected from the group consisting of a perfumery carrier and a perfumery base, and
(iii) Optionally at least one perfumery adjuvant.

14. A consumer product comprising:
a personal care active base, and
microcapsules as defined in claim 11,
wherein the consumer product is in the form of a personal care composition.

15. A consumer product comprising:
a home care or a fabric care active base, and
microcapsules as defined in claim 11,
wherein the consumer product is in the form of a home care or a fabric care composition.

16. The process of claim 1, wherein the hydrophobic material is a perfume.

17. The poly(amide-ester) core-shell microcapsule according to claim 12, wherein the poly(amide-ester) shell comprises:
optionally from 0% to 75% w/w of the stabilizer,
from 10% and 90% w/w of the acyl chloride,
from 0 to 50% w/w of the amino compound A,
from 1% to 40% w/w of the amino compound B, and
from 1 to 60% w/w of the polyol,
based on the total weight of the shell.

18. A consumer product comprising:
a personal care active base, and
the perfuming composition as defined in claim 13,
wherein the consumer product is in the form of a personal care composition.

19. A consumer product comprising:
a home care or a fabric care active base, and
the perfuming composition as defined in claim 13,
wherein the consumer product is in the form of a home care or a fabric care composition.
